(19) United States
(12) Patent Application Publication
(10) Pub. No.: US 2026/0139279 A1
(43) Pub. Date: May 21, 2026

(54) Carbon Dioxide Generator, System, and Methods

(71) Applicants: Glen Babcock, Missoula, MT (US); Wendy Babcock Garrett, Missoula, MT (US)

(72) Inventors: Glen Babcock, Missoula, MT (US); Wendy Babcock Garrett, Missoula, MT (US)

(21) Appl. No.: 19/303,288

(22) Filed: Aug. 18, 2025

Related U.S. Application Data
(63) Continuation of application No. PCT/US2024/016560, filed on Feb. 20, 2024.
(60) Provisional application No. 63/447,000, filed on Feb. 20, 2023.
(60) Provisional application No. 63/529,998, filed on Jul. 31, 2023.
(60) Provisional application No. 63/545,063, filed on Oct. 20, 2023.
(60) Provisional application No. 63/545,324, filed on Oct. 23, 2023.

(51) Int. Cl. C12P 1/02; C12M 1/00; C12M 1/107; C12M 1/12; C12R 1/645

(52) U.S. Cl. CPC C12P 1/02; C12M 21/04; C12M 25/02; C12M 29/04; C12M 37/04; C12R 2001/645

(57) ABSTRACT

An apparatus, system, and method for CO₂ production and delivery is provided. The generator apparatus supplements or supplies CO₂ where it is desired, such as to plants growing in space, particularly within an environment control system (ECS). The various embodiments of the generator apparatus are designed to withstand outer space storage, travel, and environmental impacts on a prepared mycelial mass. One example embodiment comprises dual containers wherein an inner container holds the prepared mycelial mass and an exterior container further protects the interior container and collects and sequesters condensation. In this example each container has a least one wall having a passive gas filter which permits the transfer of CO₂ out of, and oxygen in, through the first wall and out of the second wall and into an external environment. The generator may be aided by methods and system components including a cover, protective barrier, and case.

### CROSS-REFERENCE TO RELATED APPLICATIONS

This patent application is a bypass continuation of the currently pending, PCT/US2024/016560 filed on Feb. 20, 2024 which claims priority under 35 U.S.C. § 119(e) to U.S. Patent Application Ser. No. 63/447,000, entitled “CO2 Generating Apparatus”, and filed on Feb. 20, 2023, which application is now pending. This patent application also claims priority under 35 U.S.C. § 119(e) to U.S. Patent Application Ser. No. 63/529,998, entitled “CO2 Generating Apparatus and Methods”, and filed on Jul. 31, 2023, which application is now pending. This application also claims priority under 35 U.S.C. § 119(e) to pending U.S. Patent Application Ser. No. 63/545,063 filed on Oct. 20, 2023 entitled “Carbon Dioxide Lure and Adhesive Trap.” This patent application also claims priority under 35 U.S.C. § 119(e) to pending U.S. Patent Application Ser. No. 63/545,324 filed on Oct. 23, 2023 entitled “Contiguous CO₂ Lure and Water Pump.” The entire disclosures of the PCT application and those provisional patent applications are hereby incorporated by reference.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON A COMPACT DISC

Not applicable.

### BACKGROUND OF THE INVENTION

### 1. Field of the Invention

This invention relates to a non-electrical carbon dioxide generating system, method, and apparatus for gas supply or supplementation in man-made environments, and more particularly to an organism-driven carbon dioxide generating apparatus, system, and methods for space applications.

### 2. Description of the Related Art

Prolonged space travel or human habitation in space or other environments which do not permit the production of food will require a replenishable food supply within inhospitable, typically harsh conditions. For deep space travel which may cover vast distances and intervals of time, plant production for food will be necessary. Efforts have been made to grow plants in space and artificial growing environments and those efforts are always adapting to optimize such growth. Aside from growing medium and water supply, plants also require sunlight, thermal control, and gas supply. In deep space, even ignoring the other requirements, there is no carbon dioxide and therefore, plants are unable to photosynthesize meaning they cannot survive. In the past, horticultural efforts have been dedicated to manned missions where the astronauts'natural exhalation of carbon dioxide provided an expected source to aid in plant growth. Future deep space missions and habitats will be unmanned or temporarily unmanned but can be producing food along the mission routes or during vacancy periods in autonomous grow chambers.

Typical carbon dioxide products used in indoor growing are mostly in the form of a pressurize cylinder. Pressurized vessels are not ideal for rocket travel due to the chance of explosion. Other forms of carbon dioxide for indoor gardening have come from the burning of propane. Once again, such combustion is not an acceptable option for a rocket. There is a need for carbon dioxide supplies, especially on unmanned missions or during station-vacancy periods.

There is little to no written research for use of microorganisms and particularly fungi for outer space CO₂ generation where CO₂ is viewed as a liability and should be limited. Prior inventions from these inventors have sought to supplement carbon dioxide to growing environments including greenhouses, grow rooms, and underwater. The entirety of the disclosures of the prior patent application families from Glen Babcock and Wendy Babcock Garrett are incorporated by this reference as though fully set forth here.

### BRIEF SUMMARY OF THE INVENTION

A non-electrical, solid state CO₂ generating apparatus with methods of construction, packaging, transport, storage, and use as well as a system of accommodations for use in a terrestrial or extraterrestrial environments, such as an Environment Control System (ECS) are provided. The carbon dioxide generator of the present invention provides a sealed container with an interior volume space or chamber to hold and protect specially chosen and treated respirating organisms. The sealed container is configured to permit air exchange between the interior volume and an extraterrestrial environment through either an air-permeable membrane, breathable material, or an air-permeable filter. A respirating organism, in this example mycelium, occupies at least a portion of the interior volume, and then a food source also occupies at least a portion of the interior volume. Depending on the container version implemented, the food source and the mycelium may be premixed or they may be separated by an internal seal applied within the interior volume. When mycelium are separated from the food source the maximum respiration potential, and therefore carbon dioxide production is delayed, hence, versions of generators that employ the internal seal are said to have delayed activation. When the user is ready to “activate” the carbon dioxide production, they will break or separate the internal seal, but it is very important that the seal around the container is not also compromised. Once the internal seal is opened, then the mycelium from one separate chamber of the interior volume can be mixed with the food source in the second separate chamber of the interior volume.

In addition to the delayed activation options, the variations of the generator structures encompass single containers or double containers, and those containers being selected from either filtered containers made of air-impervious materials or containers constructed entirely of air-permeable membranes. The use of single bags with dual filters, for example a single layer bag with a filter on opposite or adjacent walls, will have benefits in some applications. The permutations of these variations results in flexible construction and an opportunity for optimization and efficiency. In some embodiments, the respirating mass is in direct contact with a filter on the primary chamber which allows the organisms to take in oxygen and expel predictable amounts of carbon dioxide to neighboring environments. The neighboring environments ideally contain plants to take in the carbon dioxide but expel oxygen to move back through the filters. The amounts of carbon dioxide expelled are selectable and carefully controlled.

One example of a generator apparatus also includes a separate chamber with another filter and this second chamber or container occurs exterior to the primary chamber. The second chamber allows the free passage of gases but captures any water vapor that may seek to penetrate, or which escapes the interior chamber. The floating, non-constrained, filtered interior chamber is placed within a secondary exterior chamber having its own filter. For the preferred embodiment, the filter occurs on the opposite or side opposed from the interior chamber filter. The carbon dioxide generating apparatus is constructed and configured to travel within terrestrial and extraterrestrial areas and will likewise be usable on planetary and space inhabited or vacant areas. The exterior chamber can be removed or vented for pressure differential without jeopardizing the prepared growth media because of the resilience of the primary chamber. Together, the two chambers serve as a vapor equalization device and perform dual purposes including acting as a condensation collection device and a gaseous delivery device.

The preferred respirating organism is mycelium, a network of fungal threads or hyphae. The mycelia occupy the interior volume of space which may be referred to as a pocket and may be created by at least two walls folded over and sealed with at least one seal. When the pocket is filled with mycelia, a tailored food substrate which has been sanitized from any unwanted organisms or contaminants is also placed in the pocket. The combination of the mycelia and its food source are collectively referenced as a mycelial mass. In the present invention, the preferred performance ratio establishes the mycelial mass at 95% of the available apparatus volume. In one variation, the mycelial mass is placed in direct contact with a breathing port or breather patch which preferably will be covered with a filter that excludes contaminants from entering but allows gases, particularly oxygen and carbon dioxide to cross freely. The patch makes up substantially all of at least one pocket wall and the combination of the food substrate and the mycelial spawn occupies substantially all of the interior volume space. For small growing environments, the pocket mass may be less than 10 grams and still supply an impactful quantity of carbon dioxide to plants in an Environment Control System (ECS) approximately the size of a shoebox but could be larger.

The design of the present invention is scalable in at least two ways. The first mode of scalability is by adjusting the amount of mycelium in the container and the other mode is by having a larger container that once again can be regulated by varying amounts of mycelium. For smaller ECS growing chambers, a small apparatus with 2.5, 5, or 10 grams mycelium mass may be selected. For larger ECS growing environments, a larger apparatus with, for example, 12.5, 15, or 20 grams of mycelium mass could be selected, and so on-a bigger ECS would require a larger apparatus with features consistent with the present invention. Alternatively, rather than or in addition to changing the size of the apparatus, many apparatuses can be placed at various positions around a larger growing environment, ensuring consistent distribution of carbon dioxide around the environment being supplied or supplemented with CO₂. Concentration of mycelia to food substrate can also be altered to impact the production output and longevity.

The ECS will control temperature, light, and atmospheric pressure gradients. Depending on the objectives of the targeted environment, larger masses may be beneficial so long as the ratio of the container's volume and the mycelial mass supply carbon dioxide to the environment consistent with the invention's objectives. As the mycelial spawn grow and consume the food substrate, the mass may change but all or nearly all of the carbon dioxide respirated by the organisms is released through the one or more filters.

The additional, active chamber made of plastic with its own gaseous exchange filter placed around the external surface of the pocket provides additional protection for the mycelia and also protects the external environment from water vapor transfer created by the respirating organism in the pocket. The mycelia consume oxygen and food to respirate and exhale carbon dioxide. The gases flow across the filter but metabolic liquid (water) is retained, particularly between the apparatus walls. The external chamber acts as a vapor equalization device providing dual performance to collect condensation and gas delivery passage.

Embodiments of the extraterrestrial carbon dioxide pocket generator comprise an enclosed environment having an air access port, a terrestrial respirating organism, a food source tailored to the terrestrial respirating organism, the terrestrial respirating organism being placed with the food source inside of the enclosed environment such that the respirated carbon dioxide may exit the air access port. Other embodiments do not require gas exchange port because the oxygen and carbon dioxide can move through the membrane material used to construct the container.

One example apparatus has two layers of walls wherein an inner wall contains the prepared mycelial mass and an exterior wall collects and sequesters condensation. In some variations, the condensation may be sequestered away from the prepared mycelial mass, thereby stabilizing humidity, or can be used to enhance the growing environment. Each set of walls in this example have passive gas filters which permit the transfer of CO₂ out of the first wall and out of the second wall and into the external environment. In cross-section, one embodiment of the apparatus includes an external filter, an internal wall, a mycelial mass, an internal filter, and an external wall. Seals reliably and completely enclose all the layers to protect the mycelial pocket. Thus, the mycelial pocket made of a solid plastic wall and a breather patch, the pocket fully contains mycelium and a prepared growth substrate, and a second solid exterior with its own breather patch encloses the mycelial pocket after it is sealed.

The present invention includes a system of accessories for the extraterrestrial carbon dioxide generator. For storage, the respirating apparatus is placed in a fully sealed container, such as a snap-closable airtight box. During use or during pressure transitions, the respirating apparatus may be wrapped in a protective jacket with characteristics adapted for environmental challenges. In the preferred embodiment, the respirating apparatus is placed in a silicone receiver. In some instances, the respirating apparatus is placed into a foam protective cover such as a sleeve prior to placement in a silicone receiver.

An adaptation may be added to the external plastic covering, when present, which can be fitted with an altitude activated pressure relief valve to insure against rupture and aid in survivability in the vacuum of space. This relief valve will in no way compromise the mycelial growth environment housed inside the inner container.

It is an objective of the present invention to provide a multipurpose apparatus for controlling outer space storage, travel, and environmental impacts on a prepared mycelial mass.

The present invention is a gaseous delivery device. The present technology provides non-electric, safe and effective CO₂. The passive delivery system is maintenance free and provides steady predictable CO₂ for a multitude of purposes, including the use in autonomous environmental control systems.

The present invention provides plant growth facilities with vital nutrients in a controlled environment in outer space.

The present invention is usable in planetary, interplanetary, and in outer space growing environments, whether or not they are inhabited.

It is an object of the present invention to control escaping water and vapor. It is a further object of the present invention to capture water vapor or collect condensation. It is yet a further object of the present invention to provide a vapor equalization device.

The present invention utilizes small amounts of mycelia to act as effective CO₂ producing agents.

It is an objective of the present invention to provide a steady flow of, predictable, and controlled quantities of carbon dioxide. All of which are necessary in space or in controlled growing environments.

It is a further objective of the present invention to provide adaptations to demands so that the apparatus is sizeable, and available in variable concentrations and proportions.

The present invention uses mycelia as the respirating organism because it provides the purest carbon dioxide isotope available, which is utilized by plants for the most efficient photosynthetic output possible, resulting in increased plant growth and increased yields.

The present invention increases photosynthetic rates of plants which also equates to increased oxygen production by plants. In space, oxygen is a valuable commodity.

The present invention creates a multi-purpose container for long term storage or travel for mycelium by controlling its environment, collecting condensation, emitting CO₂, and stabilizing the interior humidity.

Mycelial propulsion rate of CO₂ from the present invention in low gravity and deep space is expected to stay steady as the device is a passive delivery system, meaning that it will work no matter the pressure restraints. Eventually CO₂ is emitted to the surrounding environment, much like it would be if it were being exhaled by a human being.

The present invention anticipates and is designed to accommodate mycelial organisms even if they are cloned, augmented, solar, cosmic or otherwise radiated, microwaved, emulsified, genetically altered, engineered, shifted, or modified in any way.

It is an objective of the present invention to provide a method of shielding mycelium or similar organisms in a protective barrier, like a jacket formed of a durable but malleable material such as silicone rubber.

It is a further objective to provide a method of protecting mycelium or similar organisms in a cushioned insulation jacket such as one made of foam or a similar material.

The foregoing has outlined, in general, the physical aspects of the invention and is to serve as an aid to better understanding the more complete detailed description which is to follow. In reference to such, there is to be a clear understanding that the present invention is not limited to the method or detail of construction, fabrication, material, or application of use described and illustrated herein. Any other variation of fabrication, use, or application should be considered apparent as an alternative embodiment of the present invention.

### BRIEF DESCRIPTION OF THE DRAWINGS

The following drawings and photographs further describe by illustration, the advantages and objects of the present invention. Each drawing is referenced by corresponding figure reference characters within the “DETAILED DESCRIPTION OF THE INVENTION” section to follow.

FIG. 1 is a front view according to one embodiment of the present invention showing a first container and a first filter.

FIG. 1A is a rear view of FIG. 1.

FIG. 1B is a cross sectional view of FIG. 1 taken approximately along reference line B.

FIG. 1C is a cross sectional view of FIG. 1 taken approximately along reference line C.

FIG. 2 is a first view of another embodiment of the present invention wherein two separate containers, each with a filter, are utilized.

FIG. 2A is a second view of the embodiment shown in FIG. 2.

FIG. 2B is a cross sectional view of FIG. 2 taken approximately along reference line B.

FIG. 2C is a cross sectional view of FIG. 2 taken approximately along reference line C.

FIG. 3 is a top perspective view of a sealed pocket created according to the present invention having a mycelial mass including substrate ready for insertion into a second, larger pocket.

FIG. 4 is a bottom perspective view of FIG. 3.

FIG. 5 is another, partially exploded view of the containers shown in FIG. 3 with the first pocket inserted further into the second pocket (without seams) to illustrate the construction of the interfacing containers.

FIG. 6 is another, partially exploded view of FIG. 4 with the first pocket inserted further into the second pocket, which is shown without seams, to illustrate the construction of the interfacing containers.

FIG. 7 is a bottom perspective view according to one embodiment of the present invention with the first pocket fully sealed and inserted into the second pocket which is also now fully sealed.

FIG. 8 is a top view of the embodiment shown in FIG. 7.

FIG. 9 is a front perspective view of an embodiment of the present invention which comprises a filterless, breathable container 3 with an optional, internal seal.

FIG. 10 is a front perspective view of an embodiment of the present invention which comprises a filterless, breathable container 4 with an internal seal separating organisms from food in order to accommodate delayed but sterile activation by the user.

FIG. 10A is a cross sectional view of FIG. 10 taken approximately along line A.

FIG. 11 is a front view of an embodiment of the present invention which comprises a single, filtered, container 5 constructed of an air impervious membrane and an air-permeable filter where the container is further provided with an internal seal separating organisms from food in order to accommodate delayed, but sterile activation, by the user.

FIG. 11A is a cross sectional view of FIG. 11 taken approximately along line A.

FIG. 11B is a cross sectional, perspective view of FIG. 11 taken approximately along line B but showing the internal seal 519 being opened so that the top material may be released into the bottom of the container.

FIG. 11C is a rear view of FIG. 11.

FIG. 12 is a front perspective view of FIG. 11 having the internal seal 519 in place.

FIG. 13 demonstrates the mixing of the top materials with the bottom materials after the internal seal is opened, such as is suggested in FIG. 11B.

FIG. 14 is a front view of FIG. 13.

FIG. 15 is a perspective view of the method of constructing another embodiment of the present invention which comprises a double container 6, where the internal container has a single, air-permeable filter and is constructed of an air impervious membrane provided with an internal seal separating organisms from food in order to accommodate delayed, but sterile activation, by the user while the external, filterless bag is air permeable.

FIG. 16 is a top perspective view of the resulting combination shown in FIG. 15 with the first container fully sealed and inserted into the second container which is also fully sealed.

FIG. 17 is a front view of an embodiment of the present invention which comprises a dual container 7 with dual, air-permeable filters where the internal bag is constructed of an air impervious membrane provided with an internal seal separating organisms from food in order to accommodate delayed, but sterile activation, by the user.

FIG. 17A is a rear view of FIG. 17.

FIG. 17B is a cross sectional view of FIG. 17 taken approximately along line B.

FIG. 17C is a cross sectional view of FIG. 17 taken approximately along line C.

FIG. 18 is a cross sectional, perspective view of FIG. 17 taken approximately along line C.

FIG. 19 is another view of FIG. 18 but is showing the internal seal 719 in an open position and the top material released into the bottom of the container to be mixed with the bottom material.

FIG. 20 is a front view of an embodiment of the present invention which comprises a single, filterless container 8 constructed of air permeable material.

FIG. 20A is a rear view of FIG. 20.

FIG. 20B is a cross sectional, perspective view of FIG. 20 taken approximately along line B/C.

FIG. 20C is a cross sectional view of FIG. 20 taken approximately along line B/C.

FIG. 20D is a cross sectional view of FIG. 20 taken approximately along line D.

FIG. 21A is a perspective view of an embodiment of the present invention which comprises a dual, filterless container 9 and further comprises a relief valve shown in an inward position.

FIG. 21B is another view of FIG. 21A but with the pressure rising to a level where it has inflated the outer container and has just triggered the relief valve, shown in an outward position.

FIG. 22 is a front perspective view according to one embodiment of the present invention having an airtight storage container in the unlatched position and delayed activation container with filters inside.

FIG. 23 is a front perspective view of the bag in FIG. 22 shown in isolation.

FIG. 24 is a front perspective view of the storage container in FIG. 22 shown in isolation.

FIG. 25 is a front view of FIG. 24.

FIG. 26 is a top view of FIG. 24.

FIG. 27 is a rear view of FIG. 24.

FIG. 28 is a first side view of FIG. 24.

FIG. 29 is a second side view of FIG. 24.

FIG. 30 is a schematic representation of a front view of an example container inserting into an external foam insulation cover where the details are visible as though the cover were constructed of transparent material.

FIG. 31 is a front view representing an example container shown in FIG. 30 fully inserted into an external foam insulation cover where the internal details are visible as though the cover were constructed of transparent material.

FIG. 31A is a cross sectional view of FIG. 31 taken approximately along line A.

FIG. 31B is a cross sectional view of FIG. 31 taken approximately along line B.

FIG. 32 is a perspective view of FIG. 31.

FIG. 33 is a schematic representation of a front view of another example container inserting into an external foam insulation cover where the internal details are visible as though the cover were constructed of transparent material.

FIG. 34 is a front view representing an example container shown in FIG. 33 fully inserted into an external foam insulation cover where the internal details are visible as though the cover were constructed of transparent material.

FIG. 35 is a front perspective view of the insulation cover depicted in FIG. 30 34 shown in isolation again where the internal details of the cover are visible as though it were constructed of transparent material.

FIG. 36 is a top view of FIG. 35.

FIG. 36A is a cross sectional view of FIG. 35 taken approximately along line A shown in FIG. 36.

FIG. 36B is a cross sectional view of FIG. 35 taken approximately along line B shown in FIG. 36.

FIG. 36C is a bottom view of FIG. 35.

FIG. 37 is a front view of one embodiment of the present invention showing an air permeable container with a mycelia mass shown in FIG. 20 being inserted into a protective barrier, specifically a silicone jacket where the internal details are visible as though the jacket were constructed of transparent material.

FIG. 38 is a front view representing an example container shown in FIG. 37 fully inserted into a protective barrier where the internal details are again visible as though constructed of transparent material.

FIG. 38A is a side view of FIG. 38.

FIG. 38B is a top view of FIG. 38.

FIG. 39 is a front view of another example container shown in FIG. 1 fully inserted into a protective barrier, specifically a silicone jacket where the internal details are visible as though the jacket were constructed of transparent material.

FIG. 40 is a front view of the protective barrier depicted in FIGS. 37-39 , shown in isolation.

FIG. 41 is a side view of FIG. 40.

FIG. 42 is a top view of FIG. 40.

FIG. 43 is a front view of one combination according to the system of the present invention where a container is inserted into a protective, silicone barrier, and then inserted into the airtight case, where the internal details are visible as though the structures were constructed of transparent material.

FIG. 44 is a top view of FIG. 43.

FIG. 45 is a first side view of FIG. 43.

FIG. 46 is a second side view of FIG. 43.

FIG. 47 is a rear view of FIG. 43.

FIG. 48 schematically represents one implementation of the system where the container is inserted into the cover which combination is inserted into the silicone protective barrier to create another combination.

FIG. 49 schematically represents another implementation of the system where the container is inserted into the cover which combination is inserted into the silicone protective barrier to create another combination which is then inserted into the case.

FIG. 50 is a rear view of FIG. 49.

FIG. 51 is a top view of FIG. 49.

FIG. 52 is a bottom view of FIG. 49.

FIG. 53 is a first side view of FIG. 49.

FIG. 54 is a second side view of FIG. 49.

FIG. 55 is a schematic representation of an ECS with an example container inside the ECS with plants.

FIG. 56 is a graph showing test results of three extraterrestrial CO₂ bags utilizing mycelium according to the present invention and compared with ambient air levels.

FIG. 57 is a graph showing test results of two extraterrestrial CO₂ bags (one 5 grams, one 10 grams) utilizing mycelium according to the present invention and compared with ambient air levels over a 14 day period.

FIG. 58 is a graph showing test results for a larger extraterrestrial CO₂ bag (20 grams) utilizing mycelium according to the present invention and measured over a 12 hour period.

FIG. 59 is a graph showing the correlation between available CO₂ (0-2,000 ppm) and plant production efficiency metrics, including plant survivability 591, increased yields 592, and photosynthesis maximization 593.

FIG. 60 is a graph simulating cold test duration and temperature exposure experienced by the respirating mycelia where the y-axis is in Celsius (−100 to 100 degrees) and the x-axis is time in minutes (1-60 minutes) and showing cold tolerance temperature testing performed on one embodiment of the present invention.

FIG. 61 is a front view of the present invention with a protective silicone covering after being subjected to cold temperature exposure.

FIG. 62 is a front view of the present invention from FIG. 61 shown in isolation from the silicone covering.

FIG. 63 is a back view of FIG. 62 showing mycelia after exposure to cold temperatures.

FIG. 64 is a photograph showing preparation for tissue cultures after cold temperature exposure.

FIG. 65 is a photograph of three tissue cultures taken from the mycelia shown in FIG. 63.

FIG. 66 is a photograph of the mycelia from FIG. 63 prior to tissue cultures being taken.

FIG. 67 is a photograph of the mycelia from FIG. 63 after taking tissue cultures.

FIG. 68 is a photograph of the mycelia in FIG. 67 taken 2 days after tissue cultures.

FIG. 69 is a photograph of the mycelia in FIG. 67 taken 6 days after tissue cultures.

FIG. 70 is a photograph of the first petri plate in FIG. 65 taken 2 days after the tissue culture.

FIG. 71 is a photograph of the first petri plate in FIG. 65 taken 6 days after the tissue culture.

FIG. 72 is a photograph of the second petri plate in FIG. 65 taken 2 days after the tissue culture.

FIG. 73 is a photograph of the second petri plate in FIG. 65 taken 6 days after the tissue culture.

FIG. 74 is a photograph of the third petri plate in FIG. 65 taken 2 days after the tissue culture.

FIG. 75 is a photograph of the third petri plate in FIG. 65 taken 6 days after the tissue culture.

FIG. 76 is a graph showing test results of altitude exposure during survivability testing on the present invention. The y-axis is altitude in meters (0-35,000) and the x-axis is data point measurements taken every 3 seconds (data points 1-3664).

FIG. 77 is a graph showing test results of cold temperature exposure during survivability testing on the present invention. The y-axis is temperature in Celsius (−40 to 50 degrees) and the x-axis is data point measurements taken every 3 seconds (data points 1-3641).

FIG. 78 is a graph showing test results of pressure and vacuum exposure during survivability testing on the present invention. The y-axis is pressure (−200 to 1200 hPa) and the x-axis is data point measurements taken every 3 seconds (data points 1-3626).

FIG. 79 is a graph showing test results of radiation exposure during survivability testing on the present invention. The y-axis is cosmic radiation (0-1.4 micro-Sieverts/hour μSv/h) and the x-axis is data point measurements taken at time intervals (data points 1-126).

FIG. 80 is a photograph of the mycelia extracted from a 5 gram embodiment mounted outside of the ECS during the survivability testing launch, prior to tissue cultures being taken.

FIG. 81 is a photograph of the mycelia extracted from the 5 gram embodiment mounted outside of the ECS during the launch, after tissue cultures were taken.

FIG. 82 is a photograph showing preparation for tissue cultures after survivability testing.

FIG. 83 is a photograph of five tissue cultures taken from the mycelia shown in FIG. 80.

FIG. 84A is a series of photographs showing the regrowth of the mycelia mass from FIG. 80 as they have grown at intervals of one, two, three, four, five, six, and seven days after taking tissue cultures.

FIG. 84B is a series of photographs showing a sampling of the tissue cultures in FIG. 83 as they have grown at intervals of one, two, three, four, five, six, and seven days.

FIG. 85 is a photograph of the mycelia extracted from the 10 gram embodiment mounted inside of the ECS during the survivability test launch, prior to tissue cultures being taken.

FIG. 86 is a photograph of the mycelia extracted from the 10 gram embodiment mounted inside of the ECS during the launch, after tissue cultures were taken.

FIG. 87A is a series of photographs showing the regrowth of the mycelia mass from FIG. 85 as they have grown at intervals of one, two, three, four, five, six, and seven days after taking tissue cultures.

FIG. 87B is a series of photographs showing a sampling of the tissue cultures taken from FIG. 86 as they have grown at intervals of one, two, three, four, five, six, and seven days.

FIG. 88 is a photograph showing a front view of the present invention next to an environmental control system and an airtight storage container.

FIG. 89 is a photograph showing the front view of the present invention within an environmental control system providing CO₂ to plants.

### DETAILED DESCRIPTION OF THE INVENTION

The CO₂ generator and delivery apparatus, system, and method of use and transport of the present invention offer necessary accoutrements for providing carbon dioxide in a consistent and reliable manner to a plant growth system, including one occurring in an extraterrestrial environment.

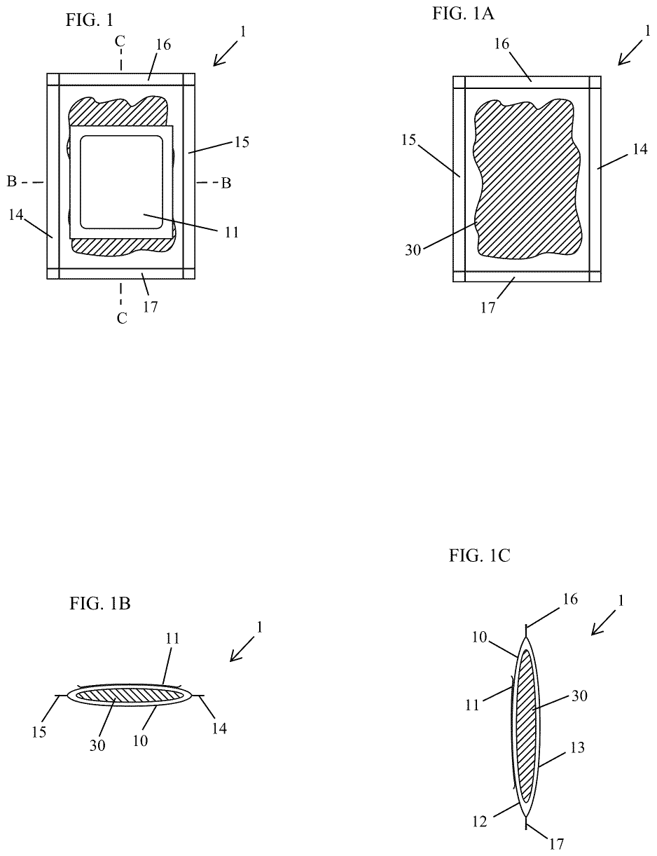

The figures represented herein are of plastic walled containers, however, the principles will apply to any container including but not limited to any enclosure, receptacle, packet, carton, envelope, vessel, bin, tub, box, or cube. FIG. 1 depicts one version of the apparatus 1 which can be quite small, in the range of a few centimeters wide by a few centimeters long. In FIGS. 1, 1A, 1B and 1C, one embodiment of the present invention may have a rugged first container 10 and a single filter 11. In FIG. 1, a filter 11 occurs on the first wall 12 (see FIG. 1C). FIG. 1A shows an opposite, second wall 13 (see FIG. 1C), without a filter, completing the pocket for the mycelial chamber 10 after seals are applied. Anywhere from 1 to 4 seals may be applied, but this example shows four seals. FIG. 1C, which is a cross section of FIG. 1 at approximately the line C, shows a first sealed end 16 occurs opposite a second sealed end 17 in the chamber 10. FIG. 1B is a cross-section perpendicular to FIG. 1C—see line B in FIG. 1. FIG. 1B demonstrates two additional seals in this example: a first long edge seal 14 opposite the second long edge seal 15. With the application of these seals, the carbon dioxide generator provides an interior volume space or chamber to hold and protect specially chosen and treated respirating organisms. The organisms combined with a manipulated and sanitized food source occupy substantially more than 50 percent of the primary interior volume.

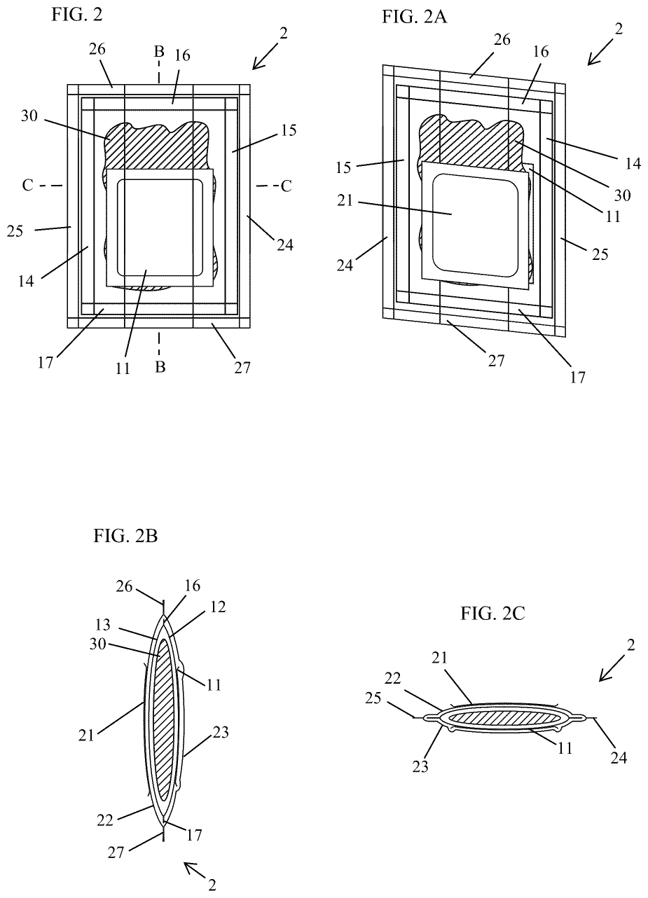

Another embodiment will include two filters made of materials with microporous capability placed on the surfaces of separate plastic, sealed pockets where the filters face opposite directions as shown, for example, in FIGS. 2 through 2C. The second container 20 with its own filter 21 holds a first container 10 which has its own filter 11. In FIGS. 2 through 2C, a first container 10 similar to the simpler container shown in FIG. 1 is inserted into a second container 20. All sides of the first container 10 are sealed and contain the mycelia and substrate (collectively referred to as a mycelial mass 30). A first sealed edge 14 occurs opposite a second sealed edge 15 while a first sealed end 16 occurs opposite a second sealed end 17 in the interior chamber 10. In FIG. 2A, it is apparent that the second container 20 has a filter 21 and also has all of its sides also sealed 24, 25, 26, and 27. The first view of FIG. 2 shows the filter 11 of the first container 10 through the transparent second wall 23 of the second container 20 (see FIG. 2B). The second view of FIG. 2A shows the filter 21 of the second container 20 on the first wall 22. Visible through the transparent second container 20 in FIG. 2A is a small amount of the rear portion of the filter 11 on the first wall 12 of the first container 10 (see FIG. 2B). Also visible through the second container 20 in FIGS. 2 and 2A is a portion of a mycelial mass 30 containing spawn and a food substrate (herein visible as the dark mass) housed in the first container 10. In this embodiment, the edges have been trimmed for compactness to be very near the seal but not so close as to jeopardize the seals.

FIG. 2B is a cross-section taken along line B in FIG. 2 and perpendicular to FIG. 2C which is taken along line C in FIG. 2. With reference to FIGS. 2B and 2C, the second filter 21 occurs on the first wall 22 of the second container 20 which amounts to an air exchange chamber. A first sealed edge 24 and second sealed edge 25 connect the first wall 22 to the second wall 23. It also shows the first sealed end 26 and second sealed end 27 on opposing sides of the exterior chamber 20.

FIGS. 2, 2A, 2B and 2C provide demonstrations of the seals that may be placed on the generator apparatus. With this sealing option, the first container is sealed and the second container is sealed, each with separate seals, which may allow the first container and chamber to move freely, or float within the second container and chamber. Alternatively, a custom container could be designed to optimize, meaning reduce, the total seals while maintaining the necessary environmental constraints. While the illustrated embodiment is using many seals, it will be desirable to reduce the total number of seals to the minimum in order to reduce fail points, or locations where human error can lead to a failure in the apparatus. Folding of a plastic wall back onto itself one or many times would reduce the needed seals and may enhance the structure of the chambers. These various views convey the free-floating nature of the first chamber 10 within the second chamber 20. The first chamber 10 is sized smaller and has no connection points to the second chamber 20 allowing it to move or float. The sizing, characteristics, and shape of the container may be adapted depending on the demands of the growing environment but regardless will be configured to contain an organism with a dedicated food supply occupying nearly the entire interior volume of the container. The two layers of walls may have advantages because they impede evaporation from the food substrate and trap any escaped moisture as condensation between the containers, and such trapped moisture can be beneficially harnessed for use by the mycelia or sequestered from the man-made environments. Condensation may become trapped between the first filter 11 and the interior of the second container 20 (see, e.g., illustration in FIG. 16). This embodiment fits comfortably in a human hand and is trimmed and ready for use.

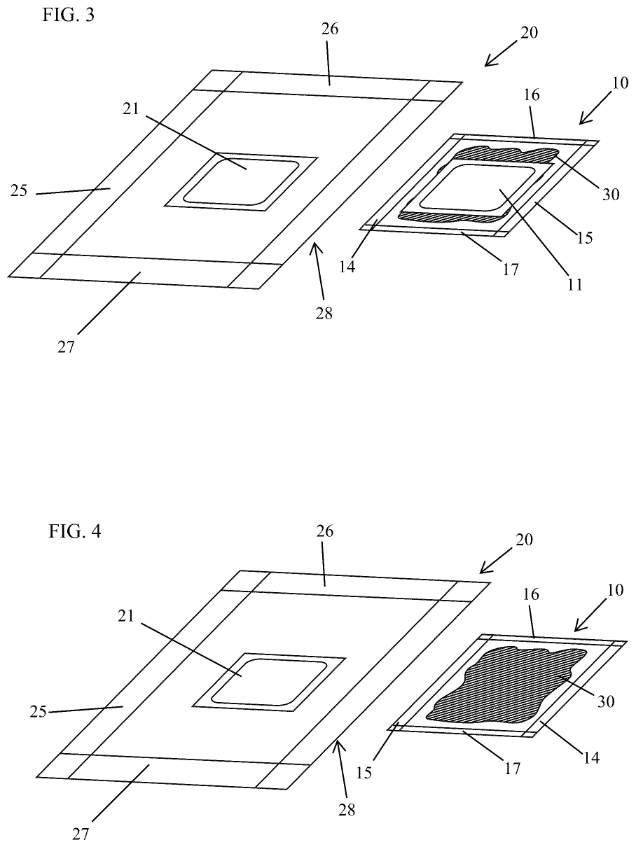

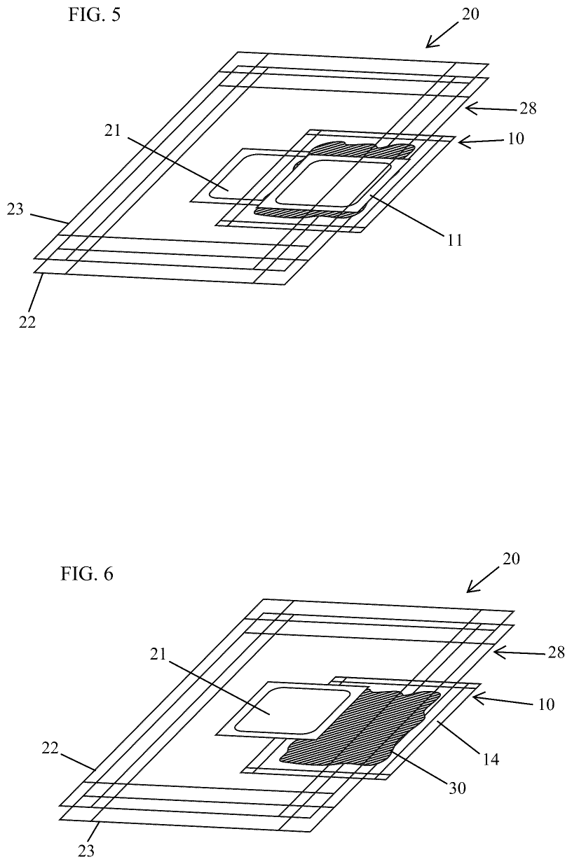

Next, FIGS. 3-6 demonstrate one method of creating a dual layer CO₂ generator and delivery apparatus according to the present invention, but other methods will become more streamlined as components may be customized to meet the desired size and dimensions and therefore reduce waste and cost. The demonstration shown in FIGS. 3-6 is only one potential mode of construction used during experimentation. The sample construction in FIGS. 3-4 depicts the following: two plastic pockets each with high-quality, gaseous interchange filters.

In some constructions, before arriving at the stage illustrated in FIGS. 3-4, one seal 15 may be added, perhaps very near the filter 11 similar to the depiction in FIG. 2. The sealing process is repeated to create a second seal 17 and then a third seal 16. Now having three total seals applied around the filter 11, a pocket is created which has one remaining opening 18 (not shown). The pocket receives the organisms and food supply according to the present invention. The respirating organism, in the preferred embodiment purified mycelial spawn 301, is mixed with a prepared food substrate 302 and the combination 30 is inserted through the opening 18 into the pocket created between the first wall 12 and the second wall 13 (see e.g., FIG. 2B). Once inserted, the final edge 14 can be sealed to enclose the organism in a pouch with a protected environment where the filter 11 permits the passage of oxygen and carbon dioxide but generally prohibits the admission of contaminants to the carefully created and optimized environment. The sealed inner pouch 10 is shown on the right side of FIGS. 3-4 . The exterior pocket 20 shown on the left side of FIGS. 3-4 is not yet sealed on one long edge as indicated by the opening 28.

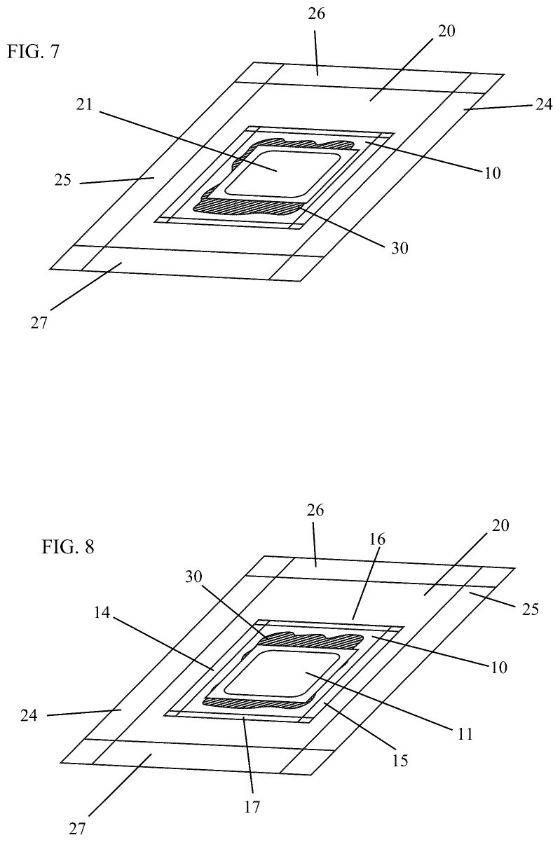

FIGS. 5-6 are schematically demonstrating the sealed inner pouch 10 being placed with the filter 11 in one direction inside the exterior pocket 20 with a filter 21 in the other direction. FIG. 5 is one view of a first pocket 10 containing a mycelial mass 30, now fully sealed, being inserted through the opening 28 of another pocket 20 visible from the bottom where the filters of the respective pockets are on walls of the pocket facing opposite directions. Depending on the shape of the final container, the filters may be placed in another arrangement such as on neighboring walls if the container has additional surfaces or sides. FIG. 6 is another view of the first pouch 10 and a top view of the second pocket 20 to illustrate the interfacing pouch and pocket prior to final sealing. The first pouch 10 is being inserted through the exploded representation of the opening 28 between the first wall 22 and second wall 23 of the second pocket 20 and the edges of the interfacing pockets are also shown.

With reference to FIGS. 7-8, the first pouch 10 is fully sealed and inserted into the second pouch 20 which is also fully sealed. Each pouch is individually sealed on the top 16, 26, bottom 17, 27, and sides 14, 24, 15, 25 for a continuous seal. The inner pouch 10 is sealed closer to the filter to allow for a slightly smaller pouch that will conveniently fit into and float without constraint within the exterior closure pouch 20. The exterior pouch 20 is sealed on a final side 24 to fully encapsulate the inner pouch 10 as shown in FIGS. 7-8 . There is a filter 21 on the second container 20 with all sides sealed 24, 25, 26, and 27. The second container 20 with its own filter 21 holds a first container 10 which has its own filter 11. All sides of the first container are also sealed 14, 15, 16, and 17 and contain the mycelia and substrate (collectively referred to as a mycelial mass 30). FIG. 8 shows the filter 11 of the first container 10 through the transparent body of the second container 20. Condensation may be trapped between the first filter 11 and the interior of the second container 20 (see, e.g., illustration in FIG. 16). FIGS. 3-8 have depicted the construction of a dual filter, dual container embodiment. A similar mode of construction may be utilized when single filters or no filters are used in dual container embodiments.

Dedicated manufacturing will optimize construction of a container and the filter, and will reduce time, costs, and waste. Pockets may be sealed and trimmed to utilize the filter section and remove excess plastic. This is only one example of construction and materials and size, but the membrane panels may be scaled as needed. Ideally, when larger containers are necessary, they will use a proportionally larger filter. Other than proportional container to filter sizing, proportional concentrations of mycelium to the mycelial mass will also be a means of controlling the needed outputs. For some embodiments, a filter is utilized by covering the majority of one wall of the container to maximize the surface area of the gas exchange.

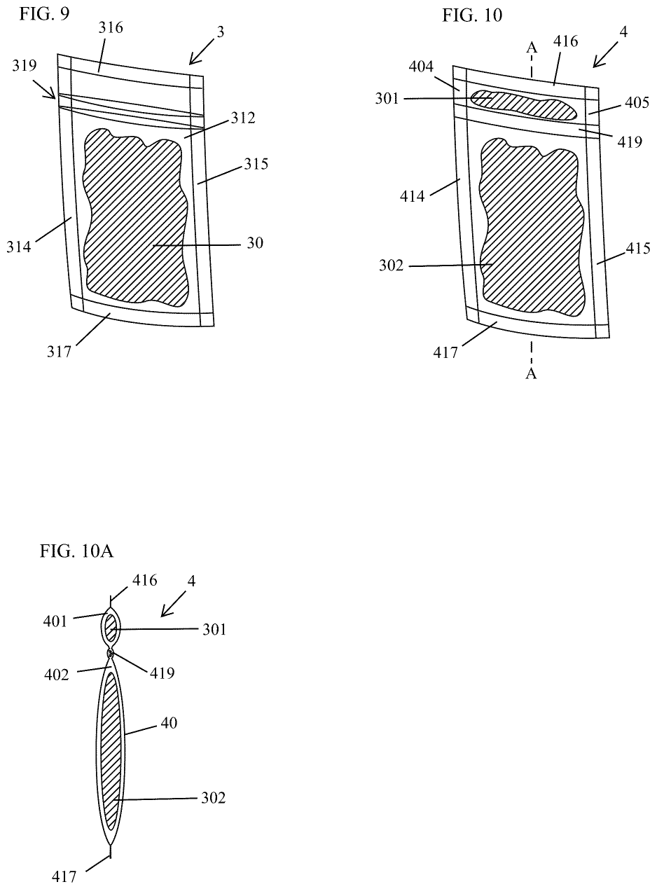

Turning to FIG. 9, another embodiment of a CO₂ generator is demonstrated. This filterless, internal seal, single bag 3 has an additional internal sealing mechanism 319 near the top seal 316 but needs no filter because the membranous material 312 of which the container is constructed breathes, meaning allows the movement of oxygen and carbon dioxide through the membrane. The mycelium is allowed access to the oxygen they need, and carbon dioxide may exit the container directly through the material. All sides of the first container are also sealed 314, 315, 316, and 317 and contain the mycelia and substrate (collectively referred to as a mycelial mass 30). In this embodiment, the internal seal 319 serves as an additional and optional protective seal.

Now, with reference to FIGS. 10-10A, another single-layer CO₂ generator 4 is depicted. FIG. 10A is a cross sectional view of FIG. 10 taken approximately along line A. In this generator version, a membrane 40 such as from polyethylene is formed into a container 4 with external side seals 414 and 415 and a finger-press internal seal 419 below the top opening 418 (not shown) which has been closed with a top seal 416 of the container 4. The bottom may also have a separate bottom seal 417 or be folded over. A food substrate 302 prepared according to the present invention is inserted into the bottom zone 402 of the container 4 below the finger-press seal 419. As shown in FIGS. 10-10A, the press seal 419 is closed so that the food substrate 302 is sequestered in the bottom zone 402 of the container 4 away from the mycelium by the external seals 414, 415, 417 and the press seal 419. At least one organism, such as activator mycelia spawn 301 is prepared and placed in the top zone 401 whereafter a final external, top seal 416 is applied to close the top zone 401.

With continuing reference to FIGS. 10-10A, the device has two distinct zones. A first zone 402 for un-inoculated substrate 302 is created by at least two external seals 414 and 415 applied to the membrane 4. The first zone 402 is closed by the manual press seal 419. A second zone 401 is created above the press seal 419. It also has at least two external seals 404 and 405 on the sides, and after the activation spawn 301 is added, a top seal 416 is created such as by a heat sealing device. The zones are separated by a Ziploc® type seal 419 and can be joined into one zone when the manual seal is opened by careful manipulation from the exterior of the container, without opening the top seal 416 or otherwise compromising the enclosed membrane 40. Depending on the mode of construction, it may be beneficial for seal 414 to be contiguous and with an uninterrupted connection with seal 404 and for the seal 415 to be contiguous and with an uninterrupted connection with seal 405.

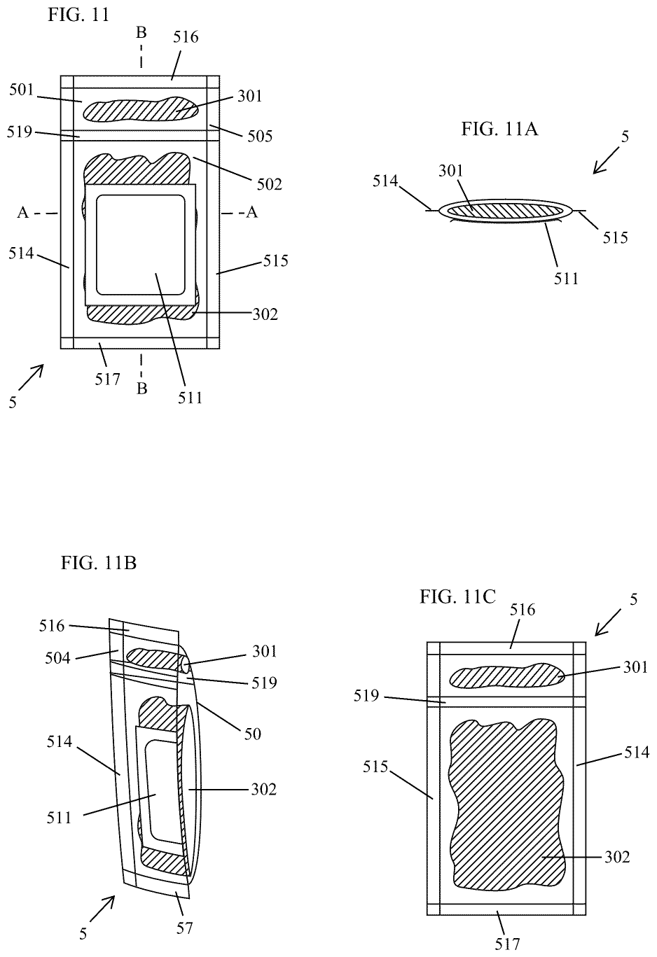

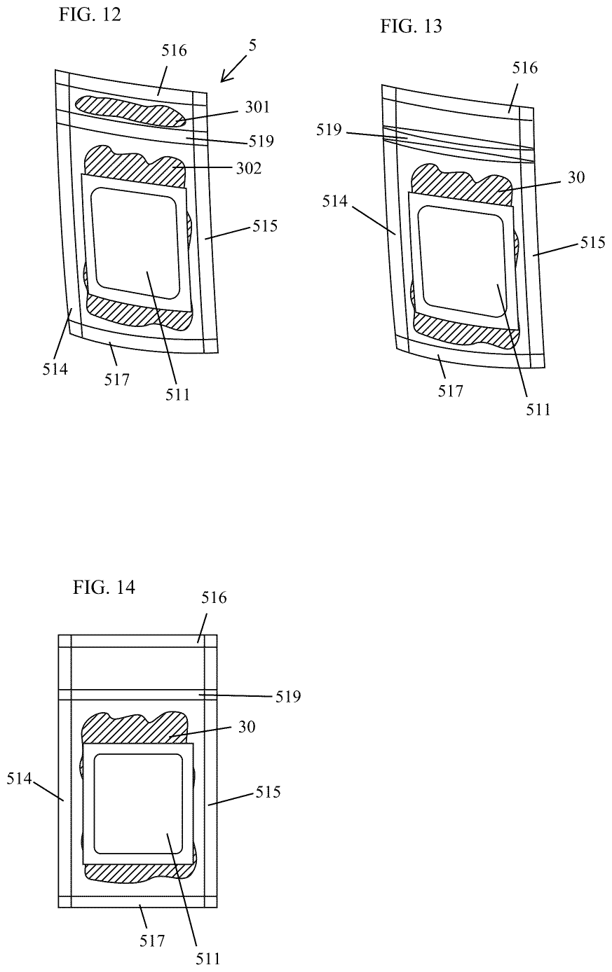

Turning to FIGS. 11, 11A, 11B, and 11C, an airtight membrane 50 such as made of polypropylene is formed into a container 5 with external side seals 514 and 515 and a finger-press internal seal 519 below the top opening 518 (not shown) which has been closed with a top seal 516 of the container 5. The bottom may also have a separate bottom seal 517, or be folded over. A food substrate 302 prepared according to the present invention is inserted into the bottom zone 502 of the container 5 below the finger-press seal 519. As shown in FIGS. 11, 11C and 12, the press seal 519 is closed so that the food substrate 302 is sequestered in the bottom zone 502 of the container 5 by the external seals 514, 515, 517 and the press seal 519. At least one organism, such as activator mycelia spawn 301 is prepared and placed in the top zone 501 whereafter a final external, top seal 516 is applied to close the top zone 501.

As demonstrated in FIGS. 11, 11C, and 12, the two distinct zones may be combined into one as is illustrated in action in FIG. 11B. A first zone 502 has a filter that allows the movement of air but not contaminants. The first zone 502 contains un-inoculated substrate 302. In the illustrated embodiment, the first zone 502 is created by at least two external seals 514 and 515 applied to the membrane 50. The first zone 502 is closed by the manual press seal 519. A second zone 501 is created above the press seal 519. In this embodiment, the top zone 501 is airtight or substantially airtight. It has at least two external seals 504 and 505 on the sides, and after the activation spawn 301 is added, a top seal 516 is created such as by a heat sealing device. The zones are separated by a Ziploc® type seal 519. FIGS. 13-14 depict the zones after mixing, where the substrate and mycelia are joined into one zone after the manual seal 519 is opened as is illustrated in FIG. 11B. The opening of the internal seal must occur from the exterior of the container through careful manipulation. Without opening the top seal 516 or otherwise compromising the enclosed membrane 50, the mycelium 301 mixes into the bottom zone 502 with the growth substrate 302 to form a mycelial mass 30. After mixing, the mycelium commence the creation of CO₂ which will move out of the filter 511.

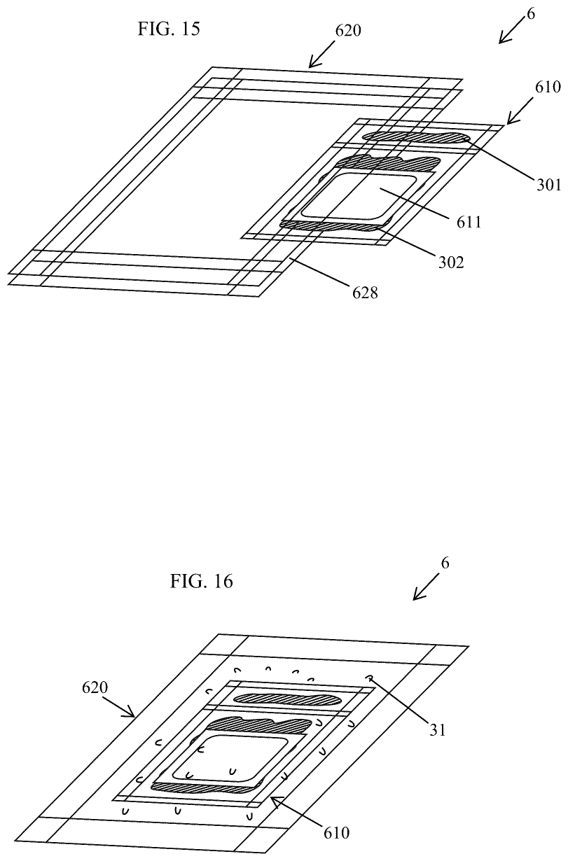

FIGS. 15-16 illustrate the construction of one example of a single filter, dual membrane CO₂ generator 6 where the internal bag 610 has a filter 611 and may be of constructions similar to the container 5 illustrated in FIGS. 11-14 . FIG. 15 schematically represents the bag 620 exploded and the internal bag 610 inserting through the opening 628. This resulting combination 6 shown in FIG. 16 has a single filter, internally sealed, delayed activation bag (see container 5) enclosed in a filterless liner 620. Condensation is expected to become trapped in various implementations of the CO₂ generator. In FIG. 16, condensation 31 is depicted to show how droplets become visibly trapped between the interior bag 610 with the single filter 611 and the interior of the second container 620.

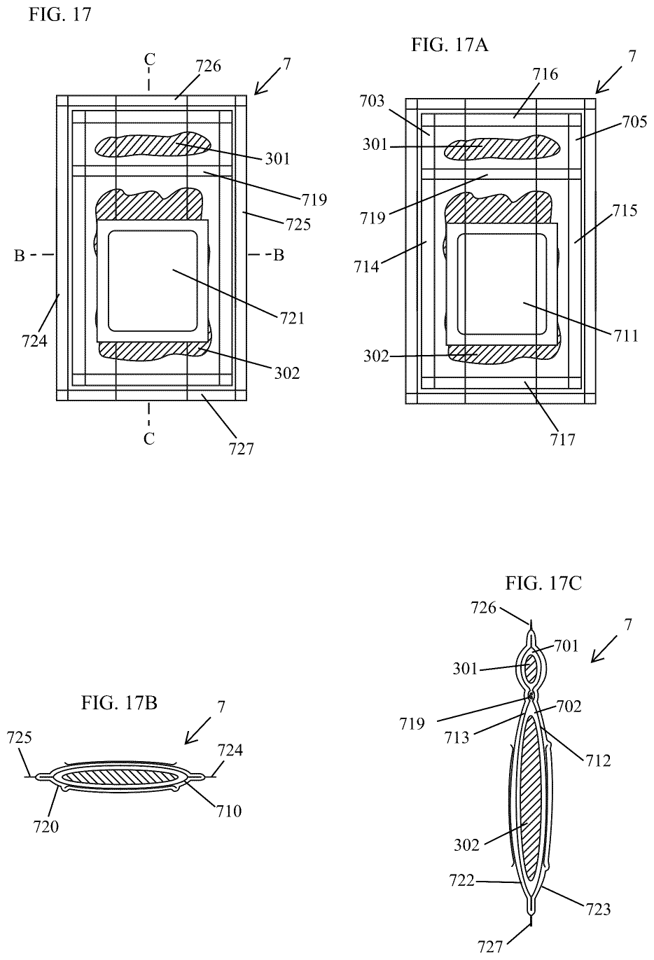

FIGS. 17-19 illustrate the construction of one example of a dual filter, dual membrane CO₂ generator 7 where the internal bag 710 has a filter 711 and delayed activation with construction which may be similar to the container 5 illustrated in FIGS. 11-14 . This combination 7 shown in FIGS. 17-19 provides two containers, each with their own filters, ideally facing opposite directions. The extra bold, vertical lines make apparent which bag is interior and which is exterior. FIG. 17 shows a first view of the combination 7 where the external bag filter 721 is visible. FIG. 17A shows a second view of the combination 7 where the internal bag filter 711 is visible through the transparent wall of the exterior bag. In this embodiment the respective filters are made of materials with microporous capability, for example filter constructed of Tyvek®. The second container 720 with its own filter 721 holds a first container 710 which has its own filter 711. All sides of the first container 710 are sealed and contain the mycelia 301 separate from the substrate 302 by internal press seal 719. A first sealed edge 714 occurs opposite a second sealed edge 715 while a first sealed end 716 occurs opposite a second sealed end 717 in the interior chamber 710. In FIG. 17, it is apparent that the second container 720 has a filter 721 and also has all of its sides are also sealed 724, 725, 726, and 727. Also visible through the transparent second container 720 in FIGS. 17 and 17A are the upper spawn 301 and the lower food substrate 302 (herein visible as the dark mass) housed in the first container 710. In this embodiment, the edges have been trimmed for compactness to be very near the seal but not so close as to jeopardize the seals.

FIG. 17B is a cross-section taken along line B in FIG. 17 and perpendicular to FIG. 17C which is taken along line C in FIG. 17. With reference to FIGS. 17B and 17C, the second filter 721 occurs on the first wall 722 of the second container 720 which amounts to an air exchange chamber. A first sealed edge 724 and second sealed edge 725 connect the first wall 722 to the second wall 723. It also shows the first sealed end 726 and second sealed end 727 on opposing sides of the exterior chamber 720. FIGS. 17B and 17C provide demonstrations of the seals that may be placed on the generator apparatus. With this sealing option, the first container is sealed and the second container is sealed, each with separate seals, allowing the first container and chamber to move freely, or float within the second container and chamber. Alternatively, a custom container could be designed to optimize, meaning reduce, the total seals while maintaining the necessary environmental constraints. While the illustrated embodiment is using many seals, it will be desirable to reduce the total number of seals to the minimum in order to reduce fail points, or locations where human error can lead to a failure in the apparatus. Folding a plastic wall back onto itself one or many times would reduce the number of needed seals and may enhance the structure of the chambers. These various views convey the free-floating nature of the first chamber 710 within the second chamber 720. The first chamber 710 is sized smaller and has no connection points to the second chamber 720 allowing it to move or float. The sizing, characteristics, and shape of the container may be adapted depending on the demands of the growing environment but regardless will be configured to contain an organism with a dedicated food supply occupying nearly the entire interior volume of the container. The two layers of walls may have advantages because they impede evaporation from the food substrate and trap any escaped moisture as condensation between the containers, and such trapped moisture can be beneficially harnessed or sequestered. Condensation may become trapped between the first filter 711 and the interior of the second container 720 (see, e.g., illustration in FIG. 16). As one example of scale, this embodiment fits comfortably in a human hand and is trimmed and ready for use.

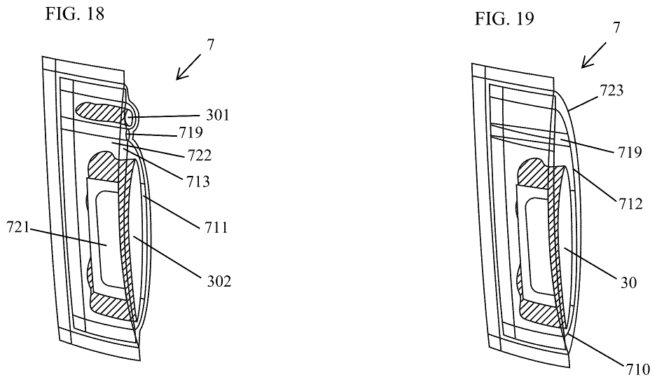

FIGS. 18-19 are perspective cross sections taken around line C in FIG. 17 and these images depict the activation steps of the delayed activation, dual filter, dual chamber generator embodiment 7. FIG. 19 depicts the zones after mixing, where the substrate and mycelia are joined into one zone after the manual seal 719 is opened as is illustrated in FIG. 18. The opening of the internal seal must occur from the exterior of the container through careful manipulation. Without opening the top seal 716 or otherwise compromising the enclosed membrane 712, 713, the mycelium 301 mixes into the bottom zone 702 with the growth substrate 302 collectively referred to as a mycelial mass 30. After mixing, the mycelium commence the creation of CO₂ which will move out of the filter 711 into the second container 720 and then out of the second filter 721 into the external environment.

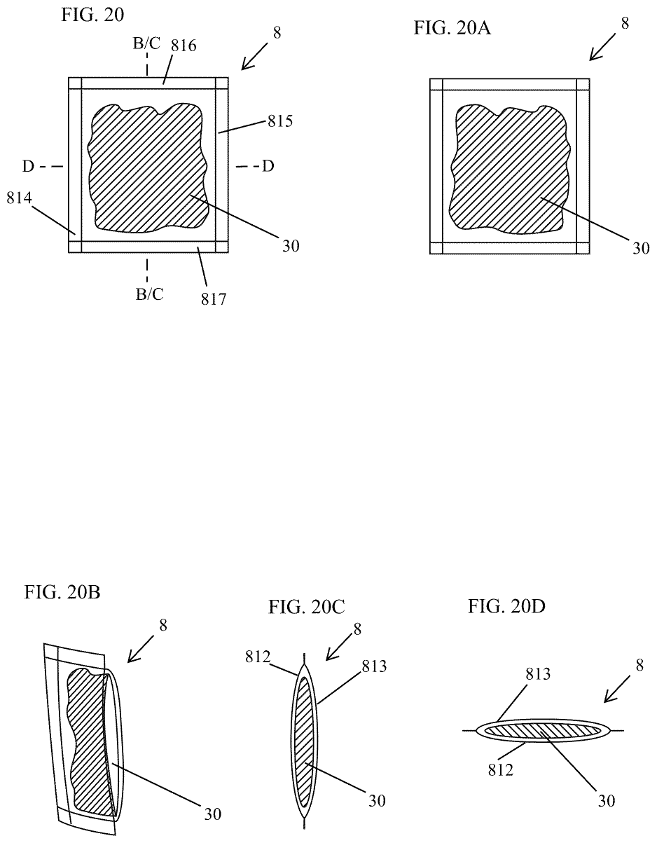

A simple, filterless carbon dioxide generator 8 is shown in FIGS. 20 and 20A through 20D. FIG. 20 is a front view and FIG. 20A is a rear view. FIGS. 20B and 20C are perspective and vertical cross sections taken around line B/C in FIG. 20. A horizontal cross section taken around line D in FIG. 20 is shown in FIG. 20D. This generator 8 is constructed of a polyethylene membrane 812, 813, but any sturdy material which is permeable to the movement of oxygen and carbon dioxide may be used. This embodiment includes a first sealed edge 814, a second sealed edge 815, a first sealed end 816 and an optional second end seal 817, alternatively being provided with a fold instead of a seal. The desired membrane material has characteristics that permit the movement of gases across the membrane but prohibit the movement of contaminants or larger particles like water across the membrane. The use of the breathable polyethylene material container obviates the need for any filters or extra gas exchange portals which will simplify manufacturing and keep costs down. Moreover, the mycelial spawn will thrive without risking their growth through any filter. The membrane is necessarily sealed and encloses an organism such as mycelium which offgasses carbon dioxide. As part of the organism's respiration it takes in oxygen, making it necessary for the membrane to be constructed of breathable material. This generator may be miniature in size and is versatile for small scale implementations of the present invention. However, the simplicity of the design will be preferred in larger implementations when appropriate membrane material is available. The careful preparation of the organism and the food source is conducted prior to placing the combination inside the container. The membrane may not be heat or autoclave resistant, so pre-sterilization and insertion of contents followed by the sealing of the membrane will protect the integrity of the membrane and the conditions of the mycelial growing environment. Despite its usual small volume, the generator has been shown to reliably produce carbon dioxide at levels that will positively impact plants for a duration of time. For installments of the present invention where even higher levels of carbon dioxide are desired or required, then a second generator 8 may be installed in a single growing environment.

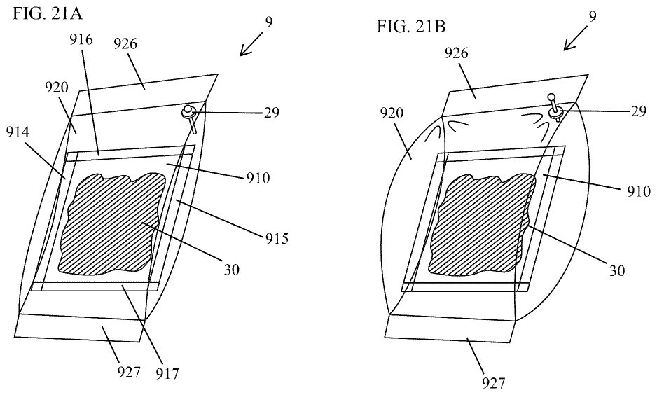

An adaptation is shown in FIG. 21A-21B . A pressure release valve 29 may be implemented in the exterior bag 920. The valve 29 will relieve and further assist with pressure changes to which the apparatus may be introduced. FIG. 21A illustrates a dual layered bag. In the illustrated embodiment, the external bag has only two seals 926 and 927 and has pleats for expansion. The pleats are clear when they are expanded by pressure build up as shown in FIG. 21B. The pressure release valve 29 will be useful no matter the number of seals employed. FIG. 21B is illustrating the precise time when the valve 29 would release because immediately after this release, the bag 920 would return to the flattened configuration. The valve 29 will have advantages when used with other external bag variations. In FIGS. 21A-21B, the interior bag 910 is illustrated as having characteristics similar to those shown in FIGS. 20 through 20D, but the exterior bag 920 and release valve 29 can be used in combination with any internal generator variation like those discussed in FIGS. 1, 9, 10 and 11. The exterior closure container may be removed or vented for pressure differential without jeopardizing the mycelial mass 30. For venting, the exterior closure can be fitted with at least one altitude activated pressure relief valve 29 to insure against rupture and aid in survivability in the vacuum of space. Again this relief valve will in no way compromise the mycelia and substrate housed inside the inner container. This is just one example of a valve but it could also be a pin-sized gap in at least one seal of the second, external container that may permit the expansion of the inner contents of the apparatus without a pressure build up which might compromise the internal, prepared environment. A small gap in the short end of the end seal 926 or 927, for example, could be filled by a moveable gasket that would exit the hole and release pressure when the pressure became too high for the external membrane to tolerate.

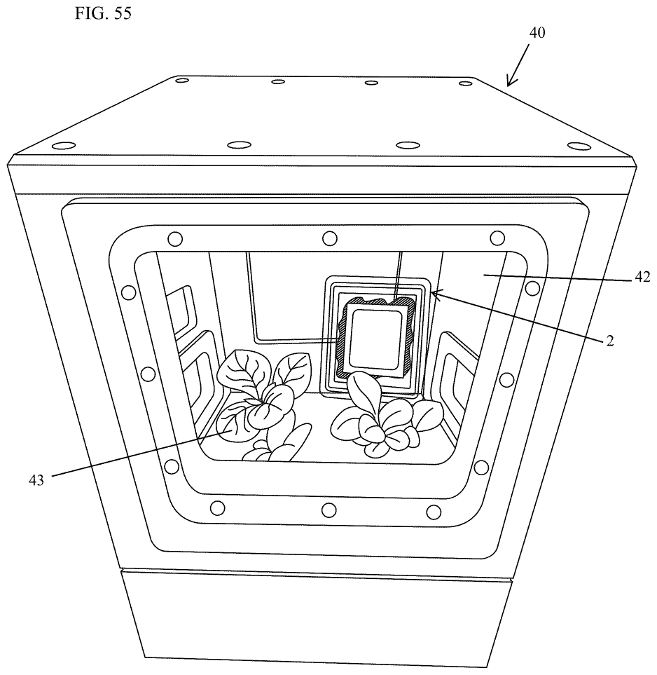

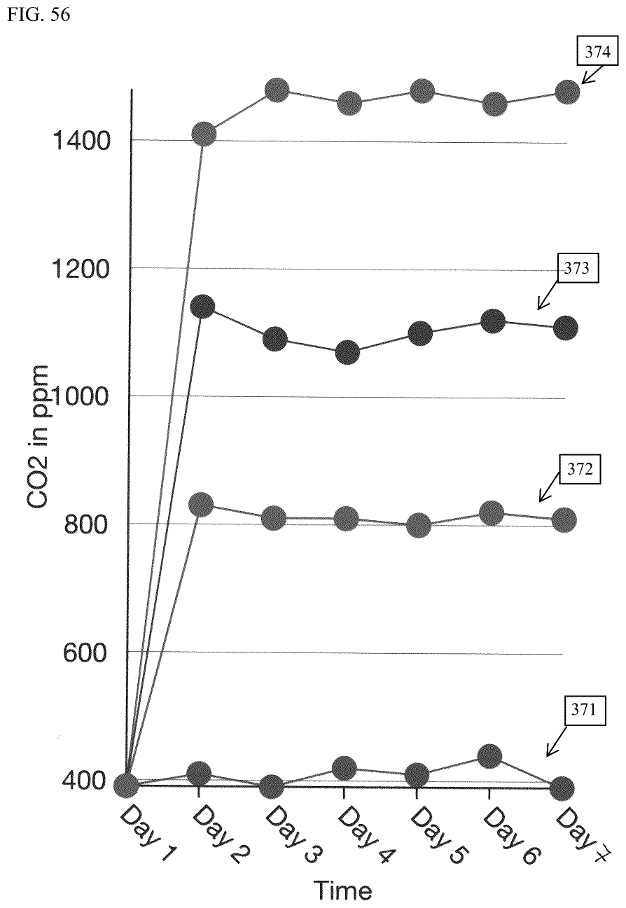

FIGS. 1-21B provide examples and details of variations, aspects, and implementations of the CO₂ generator. Sizing and weight can vary but some very small embodiments will emit a controlled and predictable carbon dioxide product into an ECS like that shown in FIG. 55. Some implementations of the generator demonstrate that the weight of the invention can be in the range of grams, for example 3.8 grams. In other examples, the container is well suited to accommodate mycelial mass weight in the range of 2.5-10 grams when the dimensions fall within a range which includes: the long edge 24 of the external container of the displayed embodiment is approximately 6.7-8.5 centimeters; the short end 26 of the external container is approximately 5.5-5.9 centimeters; and the depth of the entire apparatus in this instance is about 1.5 centimeters. An apparatus with sizing consistent with these dimensions was tested in a small ECS of approximately 3000 cubic centimeters or 0.003 cubic meters. The result of this test provided the output readings shown in FIG. 56. The size of the apparatus could be adjusted to be larger keeping with the objectives of the present invention and designed in the same fashion but customized for the given space. Alternatively, the apparatus could be utilized in multiples to service a larger growing area or ECS. Using multiples may provide additional advantages, such as being in closer proximity with particular plants exchanging gases with the apparatus. With reference to FIGS. 1B, 1C, 2B, 2C, and 20-20D, these cross-sections schematically demonstrate that the mycelial mass 30 will occupy the majority of the space within the container and will amount to nearly all of the volume of the apparatus. Though not to scale FIGS. 1B, 1C, 2B, 2C, 14 and others allow one to appreciate that the mycelial mass 30 is in direct contact with at least one of the filters, such as 11 and the various versions thereof, and occupies nearly the same end to end space as the filter itself.

For transport, storage, hibernation phases, or active supply or supplementation any of the CO₂ generator apparatuses, whether they have a single layer or dual layer, may be used in conjunction with various covers, shields, or housings. The composite system of components includes a first covering structure that will cushion and insulate the generator, a second shielding structure that will further insulate but also shield and structurally support the generator, and finally a housing closeable to create an airtight chamber for storage, delayed air exchange, or hanging during use. The present invention instructs a method of using the system in various modes and implementations, but particularly for space applications.

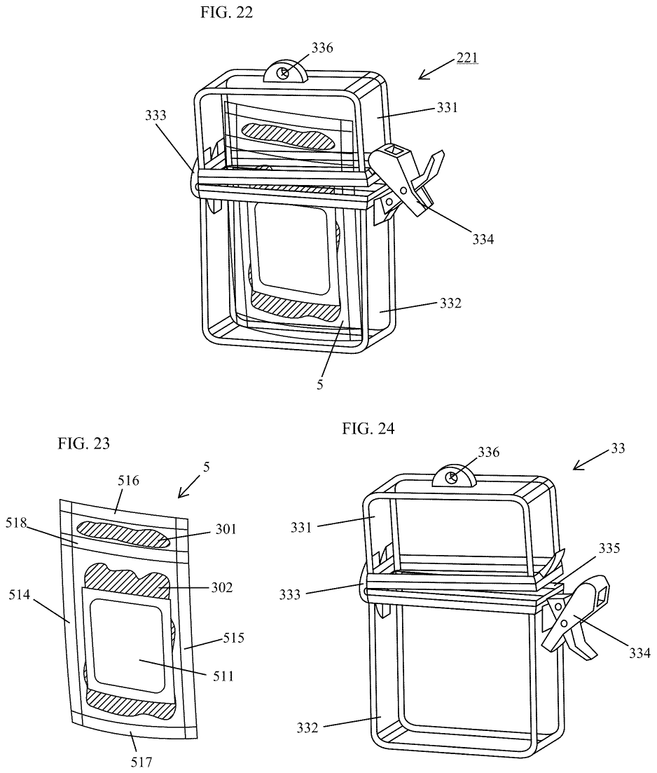

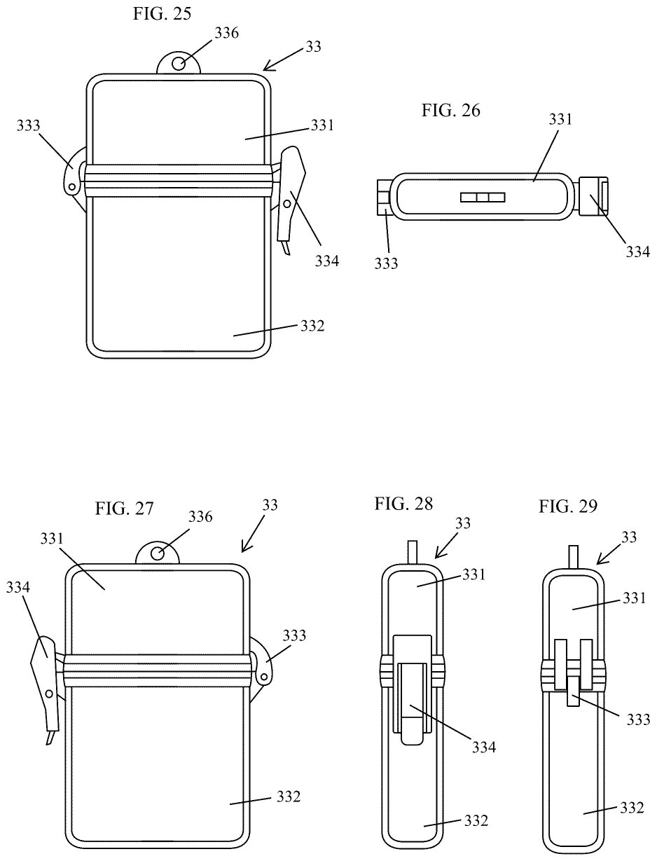

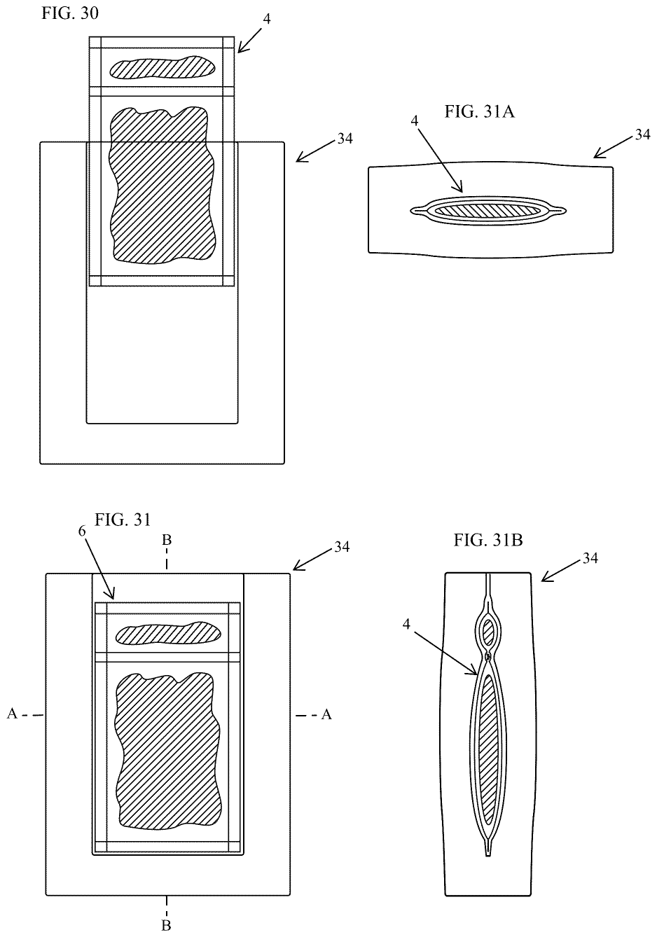

One example of a housing with an airtight chamber takes the form of an external case which is illustrated in FIG. 22. In this view, the case 33 has one of the example generators, in this instance the embodiment of the present invention which comprises a single, filtered, container 5 inserted into the case 33 to form a first combination 221 of the present invention, namely the generator and case. The depiction in FIG. 22 shows the case in an intermediate state of closure, with the latch unsealed which is one way the combination could be employed during carbon dioxide supply or supplementation. In FIGS. 23 and 24, the generator 5 and the case 33 of FIG. 22 are respectively shown in isolation. In FIG. 24, the case is shown unsealed. Further details of one preferred airtight storage case 33 is shown in FIGS. 24-29 . The front, top, rear, first side and second side views of FIGS. 25-29 illustrate the storage case in a fully sealed configuration. This airtight case 33 has a top lid 331 and a bottom receptable 332 with a hinge 333 that can be opened and then securely enclosed with a latch 334 that cooperates with the lid 331. This particular container also includes a hole 336 to receive a securing device or act as a hanger. As suggested in FIGS. 23-24, when the generator apparatus (generator 5 in this example) is to be deployed, the airtight case 33 is opened so that the generator apparatus can be placed in the ECS or similar growing environment (see FIG. 55). The top lid 331 may be opened wide as it pivots easily about the hinge 333 of the airtight case 33. The seal of the airtight case is visible as a black gasket 335 around the center of the open container. To function as a hanger, the latch 334 of the case 33 is opened to allow free movement of air past the gasket 335 while the entire device, containing the generator 5 is suspended in an environment needing CO₂ supply or supplementation. The illustrated case is transparent and hinged but could be opaque and open and close by other means, such as a two piece interference fit with multiple latches, or another suitable design.

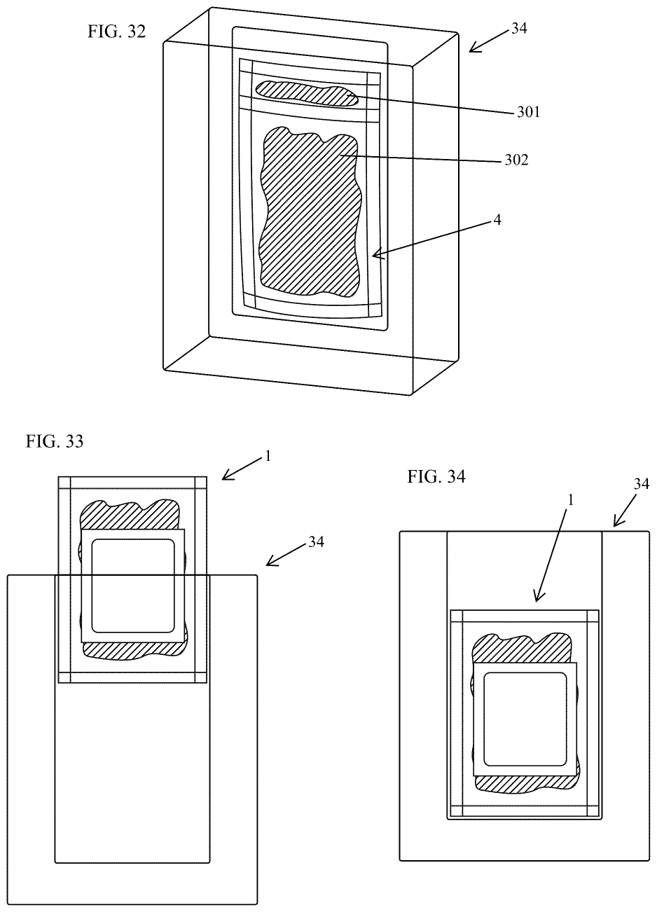

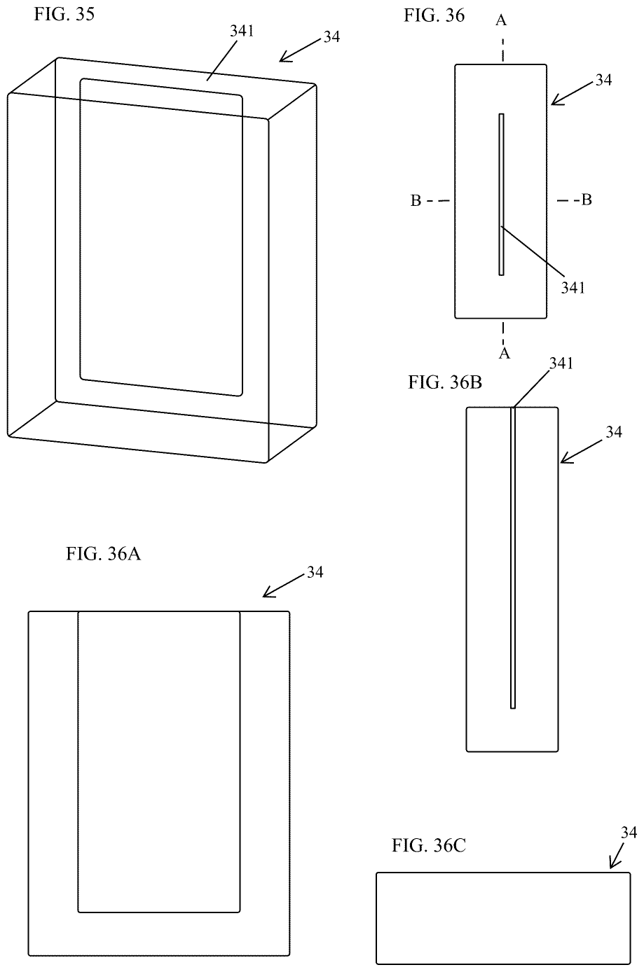

Additional external coverage or support can be employed for the apparatus, such as that shown in FIGS. 30-34 . The entire product can be fitted with an external foam insulation cover 34, a kind of thermal space blanket or cosmic shield, to aid in temperature retention and aid in survivability in the cold temperatures of low Earth orbit and deep space. For this embodiment of the present invention, the container with mycelia, generators 1 and 4 used for demonstration, may be inserted into a covering that is shaped like an envelope, or a customized pocket. One example is demonstrated in FIGS. 30-34 . Once fully inserted as demonstrated in FIGS. 31-32 and 34, the covering may provide protection or structure to the apparatus. The demonstrated example is shown in isolation in FIGS. 35-36C . In this instance, a foam material of approximately 3-5 centimeters in depth is employed. FIG. 36A shows a cross section view from approximately line A in FIG. 36 and FIG. 36B shows a cross section view from approximately line B in FIG. 36 to demonstrate an internal slot 341 to hold the carbon dioxide generator. A porous material such as that demonstrated by the foam can breathe, meaning permit gaseous exchange, and therefore could be inserted directly into the ECS growing environment while the apparatus is delivering carbon dioxide to the ECS in a manner such as that suggested herein. The illustrations of this part of the system are depicted as though the protective barrier is transparent.

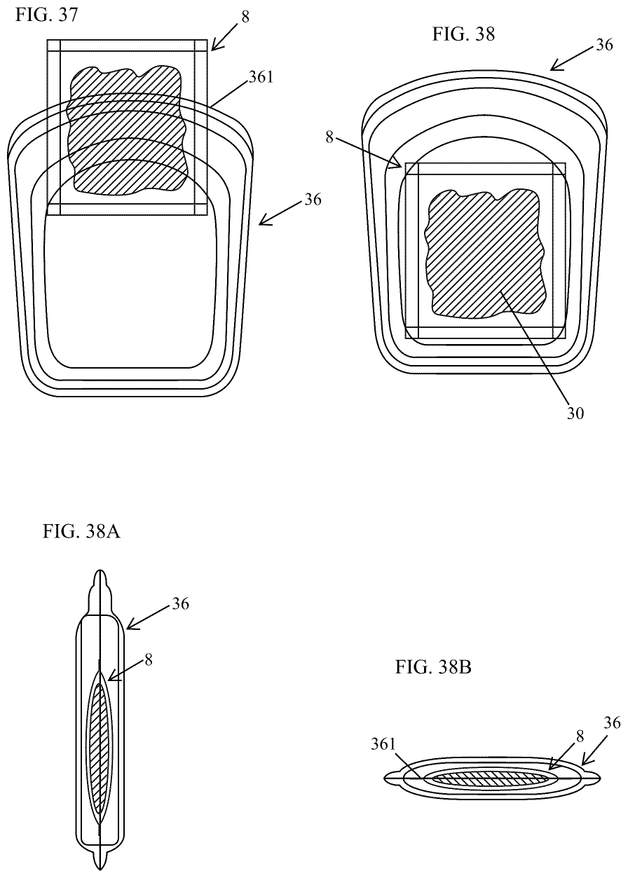

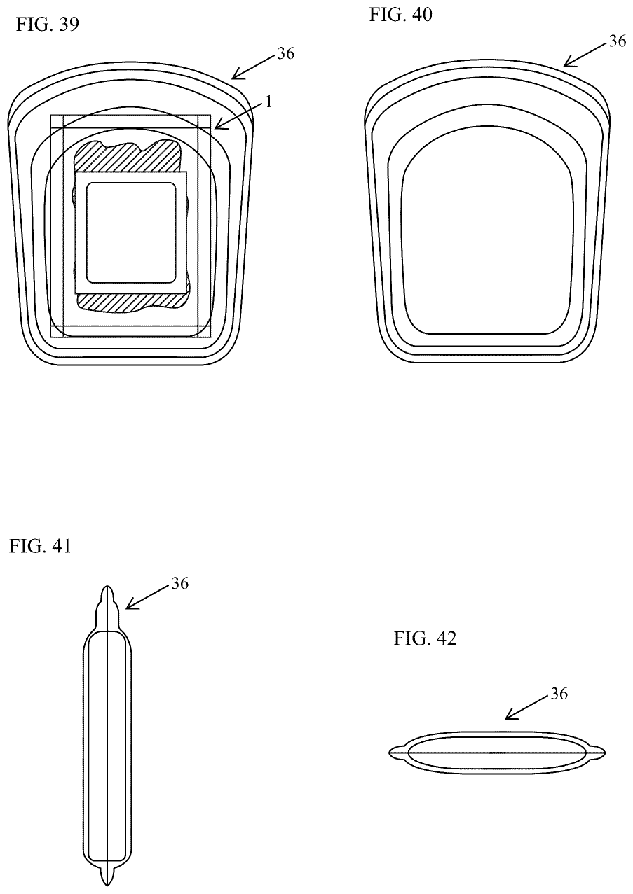

The present invention also provides for a method of protecting the mycelium or similar organisms in a protective barrier, such as a protective jacket formed of a durable but malleable material including but not limited to silicone rubber. FIGS. 37-38B provide an illustration of one generator, particularly the simple bag 8, inserting and then housed within a protective barrier 36. FIG. 37 illustrates the generator passing through an opening 361 in the barrier 36. FIGS. 38, 38A, and 38B show one example of a bag 8 fully inserted into the protective barrier 36. FIG. 38A is a side view of FIG. 38 where the protective barrier 36 is transparent so that the generator 8 is visible. FIG. 38B is a top view of FIG. 38, again, because the protective barrier 36 is illustrated as being transparent, the generator 8 is visible. The opening 361 has returned to its default, closed position once the generator 8 has passed through the recloseable opening 361. The illustrations of this part of the system are depicted as though the protective barrier is transparent. Some benefits may be appreciated from the use of a transparent material, but it is not essential to the majority of the protective functionality of the barrier 36. Another version of the generator apparatus 1 is shown inserted in the protective barrier 36 in FIG. 39.

Material such as silicone rubber is one preferred to enhance pressure resistance, provide temperature insulation, and cushion the apparatus. The nature of the material allows flexibility and malleability but also has structure that will support and shelter the important, life-supporting characteristics of the generator and the organisms housed therein. One example of a silicone jacket protective barrier 36 is shown in isolation before shielding the generator apparatus in FIGS. 40-42 . FIG. 40 is a front view of one version of a silicone jacket that may be employed to protect the mycelium as they travel to space, while FIGS. 41 and 42 are side and top views, respectively. The silicone jacket could be made from two halves having an internal cavity that connect by interference fit. Alternatively, the protective barrier 36 could be a pouch like the one illustrated with an opening at one end for inserting and removing the apparatus 1.

By employing the present invention apparatus, system, and methods mycelia and similar organisms are protected from extreme environmental conditions, including those encountered in space. In space, mycelia will be exposed to extreme environmental conditions. Cold temperatures, pressure changes, and radiation exposure are just some of the risks posed to these living organisms when located in or traveling to extraterrestrial locations. The methods of transporting living organisms like mycelia in protective compartments comprised of silicone or a similar material or layers has never before been attempted. The methods employed in the present invention were optimized for the living organisms when they were tucked into, but not sealed into the protective compartment. The testing conducted has proven that the methods of housing and transport are highly effective in protecting these living creatures in these environments.

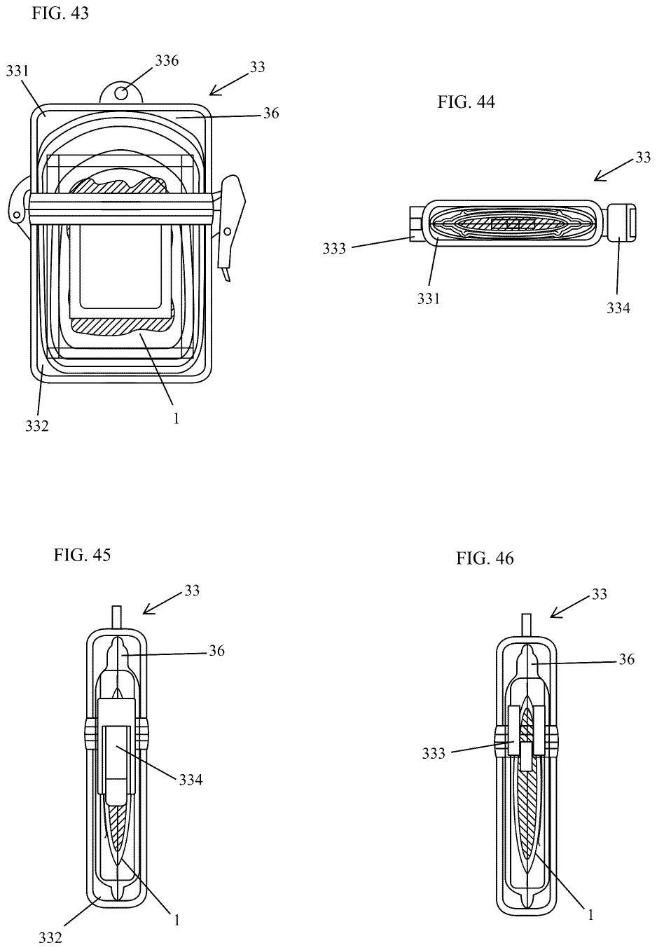

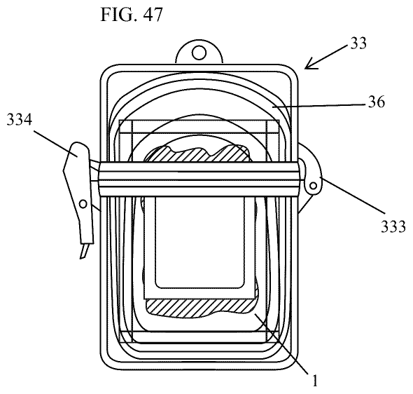

Various components of the system may be selectively employed depending on the demands of the travel, destination, and implementation conditions. The protective barrier 36 can be used in conjunction with the airtight container 33 as shown in FIGS. 43-47 . FIG. 43 is a front view of the combination while FIGS. 44, 45, and 46 are top and first and second side views, respectively. FIG. 47 is a rear view of the protective covering 36 used in conjunction with the airtight container 33 as shown in FIG. 43. In the illustrated implementation, the single filter, membrane generator apparatus 1 inserts into the protective covering 36, and then is nested in place in the airtight container 33 to make the combination that does not include a cover 34. For this version, the generator 1 is inserted through the malleable opening of the barrier 36 and then the combination is inserted into the case 33 after the latch 334 is opened so that the combination may be inserted into the bottom 332 of the case before the top 331 of the lid is closed over the combination and the latch 334 is closed to cut off air supply to the interior of the case 33.

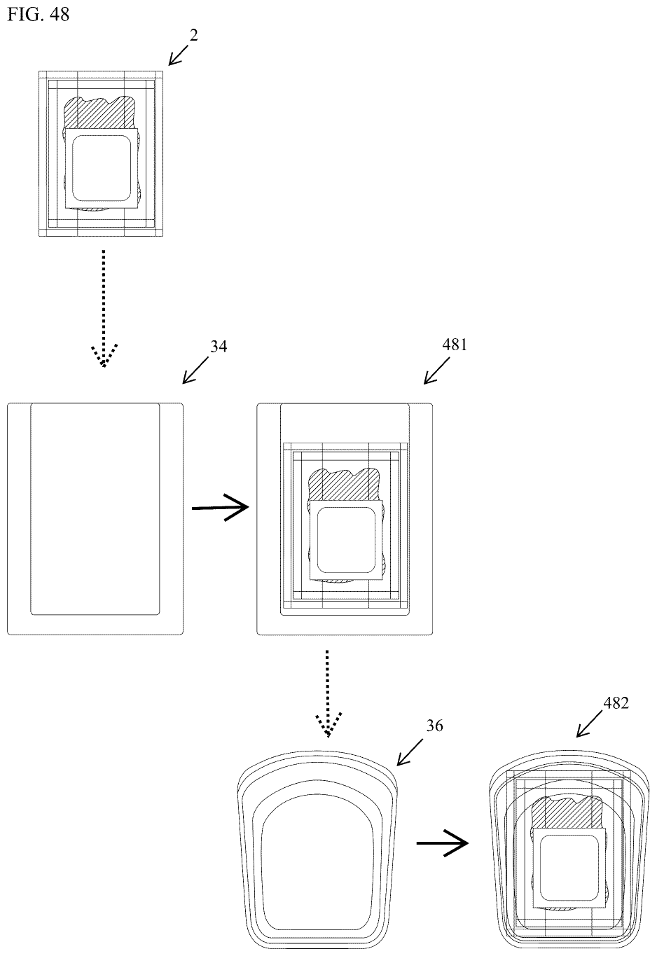

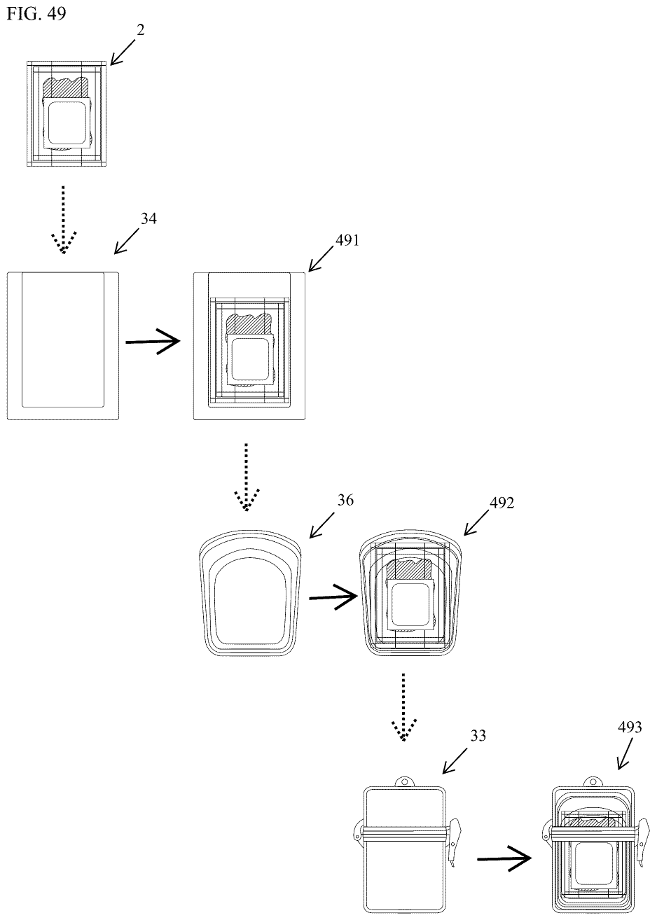

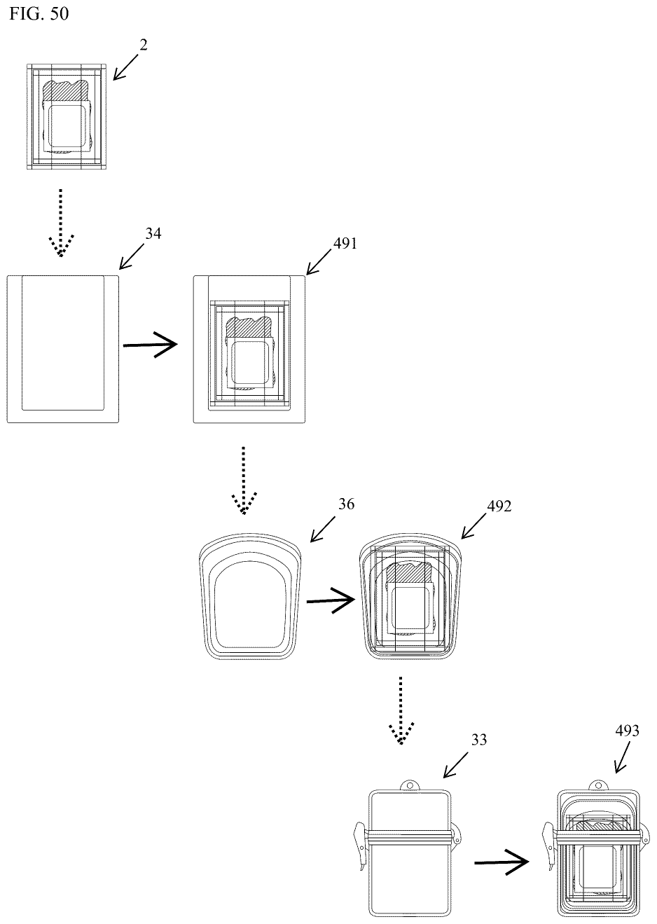

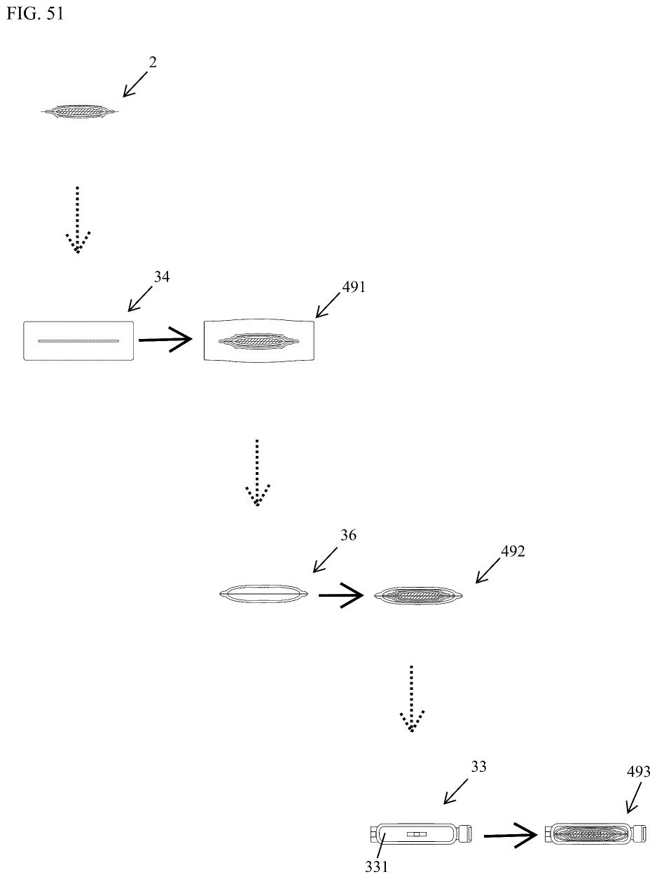

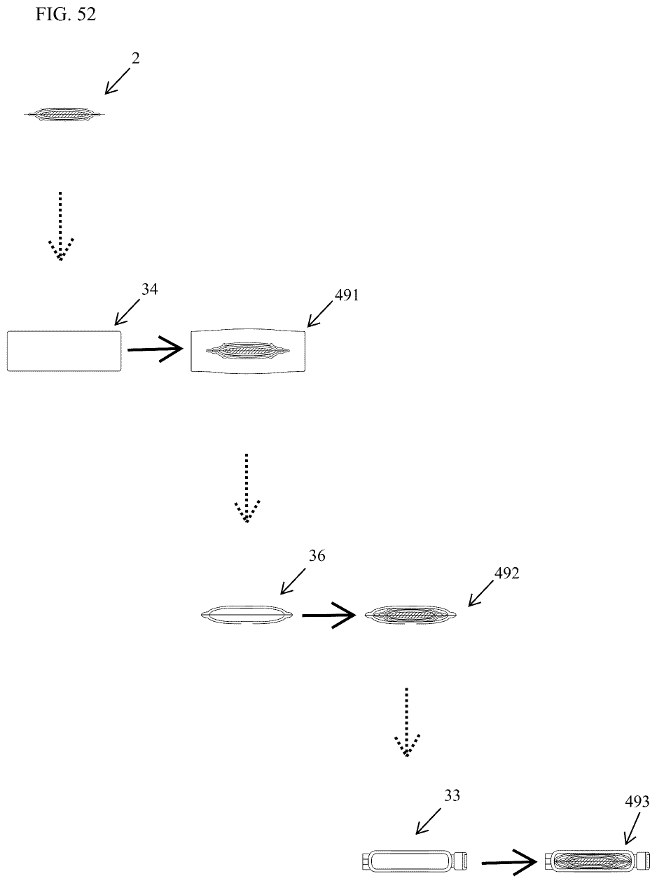

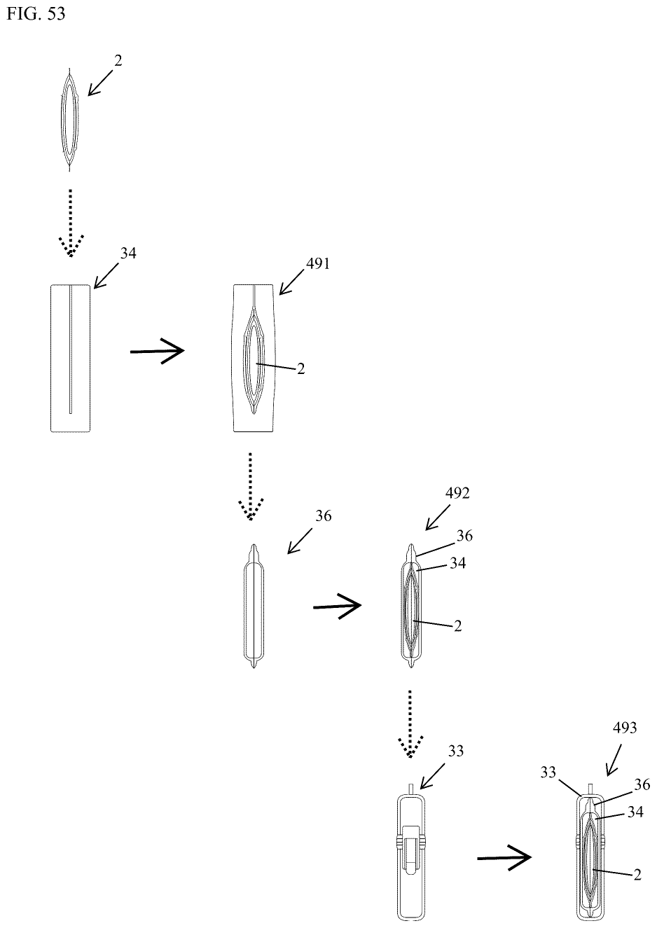

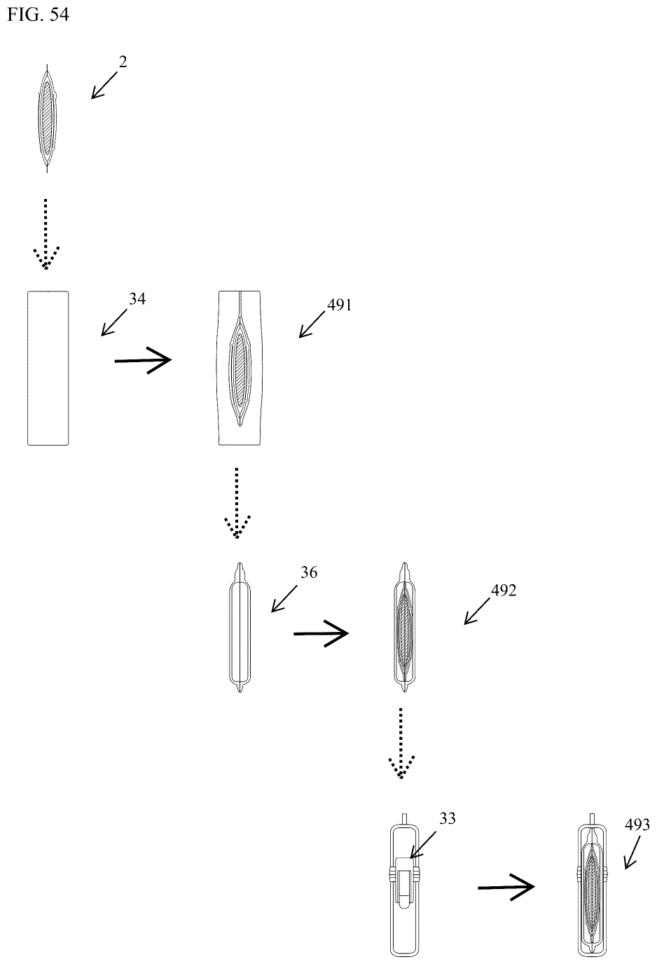

The protective barrier 36 can also be used in conjunction with only the external foam insulation cover 34 and generator as illustrated in FIG. 48. In the example illustrated in FIG. 48, the cover 34 receives the dual filter, dual membrane generator apparatus 2, as directed by the dotted arrow, to form the resulting combination 481 and both are inserted, as directed by the next dotted arrow, into the protective barrier 36 to create the combination 482 as shown. FIGS. 49-54 illustrate how the components of the system may be nested. The various configurations of the system will have benefits for storage, transport, or active carbon dioxide supply or supplementation. The additional components of the system will help to maximize the generator apparatus features. These various views depict a dual filter, dual membrane, example generator 2. Turning first to FIG. 49, the generator is represented by a dotted arrow inserting into an empty cover 34 to form the resulting combination shown at 491. In some implementations, the system may be employed so that this combination is the final combination for use. However, the first combination 491, as directed by the dotted arrow, may continue to be inserted into the protective barrier 36 to form a second combination 492. Again, in some implementations, the system may be used in this combination only. This configuration was exposed to the elements of space as described in the testing discussions below. Then, as directed by the final dotted arrow, the second combination 492 may be inserted into the case 33 to form a third combination 493. The various components are illustrated as transparent in the front and rear views of FIGS. 49 and 50, respectively. FIG. 51 shows the example generator 2 in top view and the top view of the cover 34. The resulting combination 491 is shown in horizontal cross section and is inserted into the protective barrier 36 in the next step. When the generator 2 in the cover 34 (combination 491) is inserted into the protective barrier 36, it creates the second combination 492 which is also shown in a from the top. Finally, the second combination 492 is inserted into the case 33 to create the third combination 493 which is also shown in a top view. FIG. 52 shows all of the components and combinations in bottom view and with a horizontal cross section. FIGS. 53-54 show the generator 2, the cover 34, protective barrier 36 and the case 33 are shown in either vertical cross section or side elevation view. The combinations 491, 492, and 493 are displayed in various states of tear away and cross section illustration in order to expose the nesting in the various components in the combinations.

Desirably, the material utilized for the external support, including but not limited to the foam insulation jacket and/or silicone jacket, has characteristics to assist the apparatus in withstanding large pressure changes such as those experienced when leaving or entering the Earth's atmosphere. Testing has shown that the covering may have additional properties beneficial for harsh conditions encountered during space travel or terrestrial areas with extreme climates. The preferred covering may have properties including but not limited to being breathable, insulating, radiation resistant, shock absorbing, and deformable, or expandable. The external support is demonstrated as a foam pocket without the final end being sealed, but the external support may be fully sealed as well.

A sectioned chamber container embodiment could be employed in an alternative version of the present invention. The mycelial mass 30 may be housed in the lower portion of a small bag and an external clip would optimally span the entire horizontal surface between the mycelial mass 30 and the filter. The clip separates the respirating mycelial mass from the air filter on the opposite side from the clip. To implement this version of the invention, the clip would be removed to allow the mycelial mass 30 to have access to the filtered, gas exchange port. Once unclipped, this version may be folded or manipulated to meet the demands of compact usage areas.

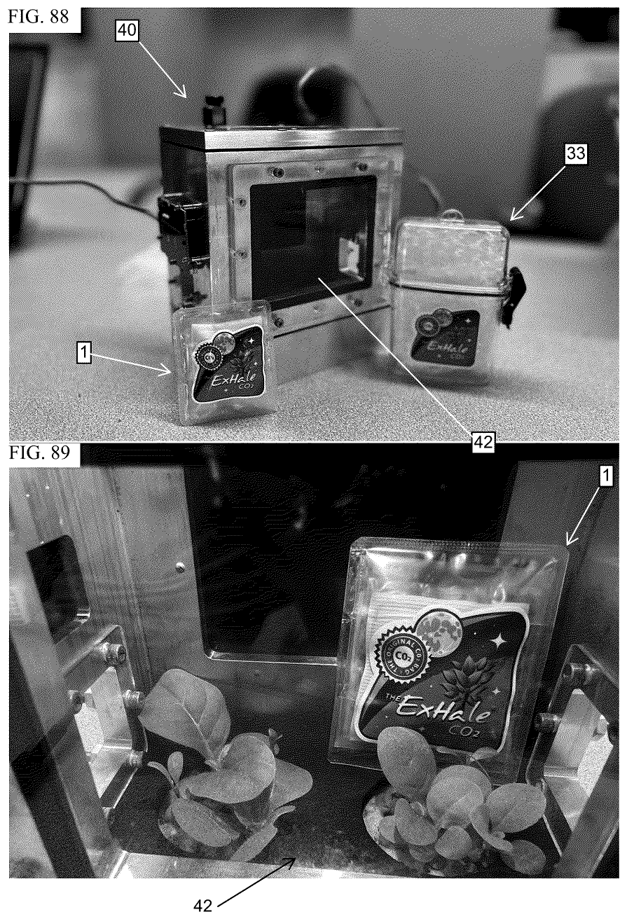

The impressive, controllable, and predictable nature of the present apparatus has been demonstrated through testing. One environmental control system (ECS) 40 similar to the one illustrated in FIGS. 55 and 88-89 was used during testing. The unit is also referred to as the FAU CubeSat HAB Payload which was launched and was subjected to the elements of space. A 5-gram generator was stored in a cover 34 within an insulated silicone pouch 36 and mounted on the external wall of the ECS 40 (not shown) in order to test the full effects of the space flight. Another generator with 10 grams of mycelium was installed inside the ECS 40. During flight, the 10-gram bag was held within the left wall of the ECS 40 to provide the plants inside with the CO₂ that is needed to keep plants alive in space. In some implementations, the ECS 40 has an internal chamber 41 (not shown) within the side wall to hold the generator apparatus of the present invention 1 and a grate (not visible) may separate the chamber 41 from the internal space 42 of the ECS. The grate will still allow CO₂ to flow from the generator 1 to the plants within the main cavity 42 of the ECS 40. In the illustrated configuration, the device 1 is placed directly within the main cavity 42 of the ECS 40 where the plants are growing. The plants 43 and the generator 2 are visible through a glass screen at the front of the enclosed ECS. The ECS 40 has internal controls operating to maintain a consistent growing environment such as keeping pressure at 1 atmosphere. The ECS will also be able to somewhat modulate temperatures and radiation exposure. For example, during testing the ECS internal temperatures ranged from 14.81 to 26.08 degrees Celsius while external temperatures ranged from 51 to −38 degrees Celsius. See FIG. 77.

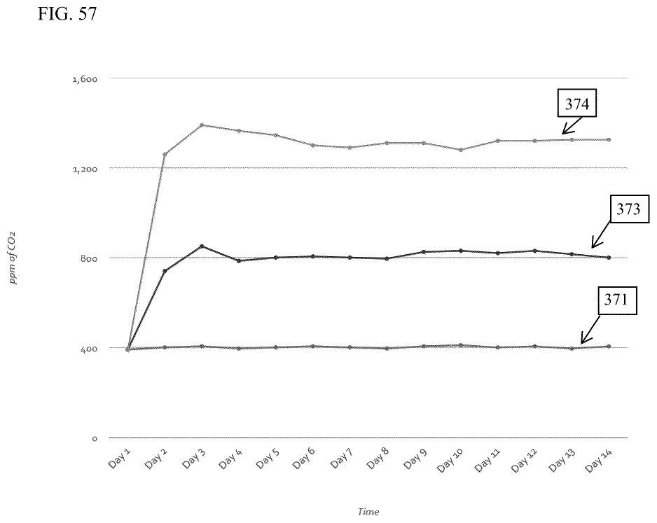

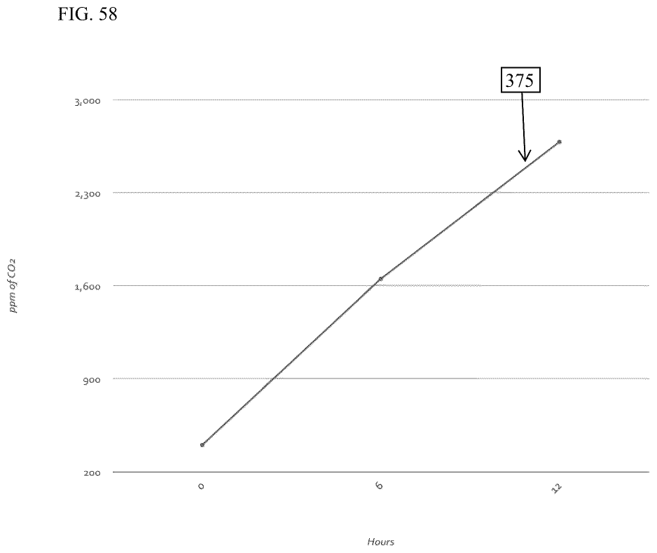

Because early ECS tests were done in a small autonomous growth chamber or micro growing chamber, a small generator 2 like that shown in FIG. 55 was designed and tested. For example, FIG. 56 is a graph entitled “Mycelial CO₂ Bag Analysis” showing test results of three CO₂ generators utilizing mycelium according to the present invention. The x-axis is time in days, marking out seven days. The y-axis shows measurements of carbon dioxide levels in units of parts per million (ppm) having lines demarcating 600 ppm, 800 ppm, 1,000 ppm, 1,200 ppm, and 1,400 ppm. The graphs shows the results of tests which compared ambient air levels and supplemented levels when tested with a CO₂ meter within an enclosed area having a volume comparable to a small ECS and without any plants in the chamber. Near the bottom of the graph, the control line 371 shows the ambient carbon dioxide levels hovered at approximately 400 parts per million (ppm) in the enclosed chamber without any supplementation over a period of seven days. The next line 372 illustrates that the 2.5-gram generator, also known as a space bag, enhances CO₂ levels by double those measured in the ambient air, in this example from 400 ppm for ambient air to approximately 800 ppm on days two through seven when supplemented with the 2.5-gram space bag. A 5-gram space bag test is shown by the third line 373. For the 5-gram space bag test, the CO₂ levels were demonstrated to rise to approximately 1100 ppm (700 ppm above ambient levels 371) by day two and remain in that range through day seven. Finally, the 10-gram space bag test, as shown by the fourth line 374, raised the CO₂ levels from the 400 ppm ambient levels to more than 1400 ppm within the ECS by day two and those levels remained in that range through day seven. Similarly, a CO₂ generator analysis comparing two bags was conducted and the results are shown within FIG. 57 where the x-axis is time marked in days from Day 1 to Day 14 and the y-axis is levels of CO₂ in parts per million (ppm). The test results of a 5-gram space bag shown by the middle line 373 and a 10-gram space bag shown by the top line 374 were compared with the control line 371 over a 14 day period. The 5-gram bag continued to produce around 800 ppm and the 10-gram bag continued to produce around 1,400 ppm for the duration of the test. For comparison, and as shown in FIG. 58, a CO₂ generator analysis was conducted for a 20-gram space bag over a 12-hour period. The x-axis for the graph in FIG. 58 shows time in hours while the y-axis reports carbon dioxide in parts per million (ppm). The carbon dioxide output, as shown by line 375, for the 20-gram generator demonstrates a rise in carbon dioxide from about 400 ppm at hour zero to about 2,600 ppm at hour twelve. The test results overall demonstrate that the present invention prepared according to this disclosure creates carbon dioxide supplementation that can both be controlled and predicted over a period of time. Additional testing supports the predictability of carbon dioxide nutrient supplementation that is harnessed and controlled at impactful concentrations based on amounts of spawn and food source. The preferred apparatus, no matter the final total weight, is designed to supplement carbon dioxide in a manner consistent with the graphs for a predictable period of time and as suggested by the present testing, for a period of time far exceeding the testing periods thus far.

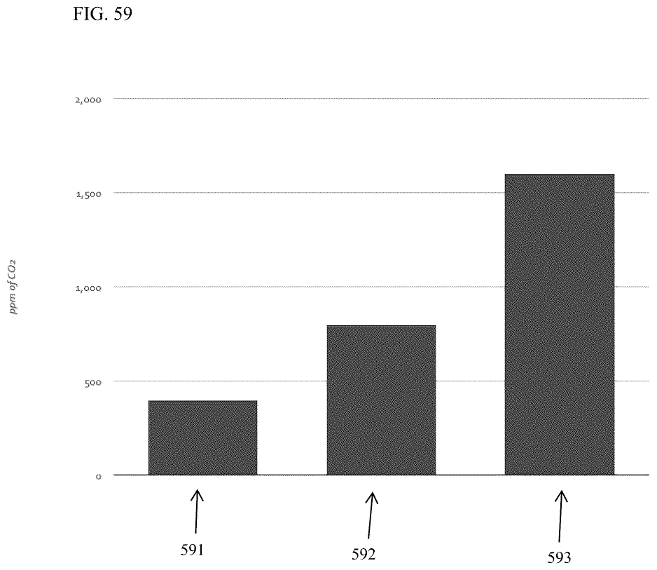

FIG. 59 is a chart showing various levels of CO₂ and the effects on plant production efficiency. The bar graph has an x-axis of plant actions, namely “Plant Survivability” at bar 591 and “Increased Yields” at bar 592 and “Maximize Photosynthesis” at bar 593 The y-axis of the bar graph shows measurements of carbon dioxide in parts per million (ppm), having lines demarcating 500 ppm, 1,000 ppm, 1,500 ppm, and 2,000 ppm. The results of this test demonstrate that when carbon dioxide rises through supplementation, plants become more efficient and productive. This shows the correlation between the amount of CO₂ exposure to plants and the plant growth efficiency. In FIG. 59, bar 591 shows that with a carbon dioxide level of about 400 ppm plants are able to survive. When carbon dioxide is increased, plants will increase their yield. The testing illustrated in FIG. 59, particularly bar 592, demonstrates that when carbon dioxide is supplemented to levels of as little as about 750 ppm, increased yields are measured; and finally as shown at bar 593 at around 1500 ppm plants will be able to maximize photosynthesis under otherwise consistent growing conditions.

Cold tolerance testing was conducted on the mycelia from the CO₂ generator when it was contained in the cover 34 and protective barrier 36. Because space based applications require exposure to extreme cold, this test was conducted to ensure viability of the mycelium when used according to the claimed methods and apparatus. As shown in FIG. 60, an earth bound cold test was performed to simulate cold test duration. The test was performed at Florida Atlantic University (FAU) prior to the space-based test. The test subjected the 5-gram generator housed within the protective barrier 36 which was closed with cable ties at the opening of the protective barrier. Alternative closure security could be used in place of the cable zip ties to provide extra security in the closure and tamper proof features to ensure the mycelium travel without alteration or damage. The 5-gram CO₂ generator stored in the protective barrier 36 was externally mounted on an ECS and placed in a deep freeze. The x-axis of the graph in FIG. 60 marks time in quarter hours. The test ran from 0 minutes to 60 minutes, plotting data at 1, 15, 30, 45, and 60 minutes.

The y-axis shows the temperatures in Celsius reached during the test with demarcations at −100 C, −50 C, 0 C, 50 C, and 100 C. During this earth-bound testing, the space bag was inserted in the deep freeze where it took about 1 minute to reach the maximum cold temperature of −80 C which it continued to be subjected to for 45 minutes before being removed from deep freeze and taking 15 minutes to return to the beginning temperature, see FIG. 60.

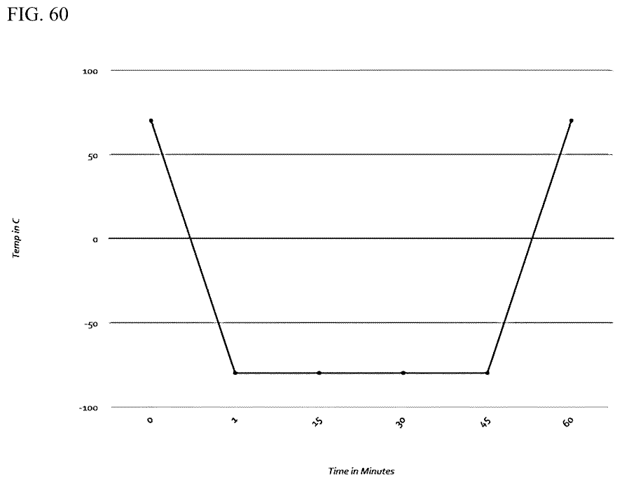

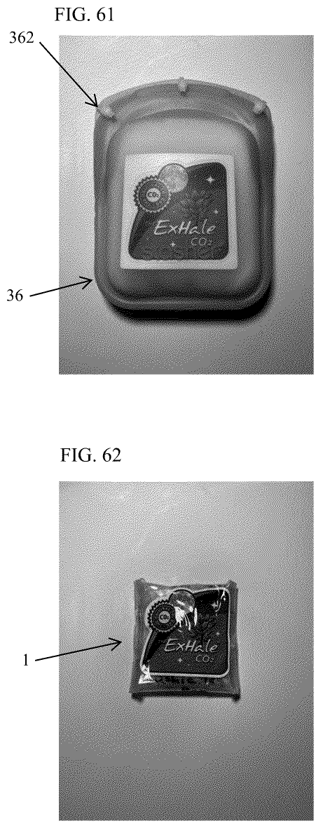

The 5-gram space bag combined with the foam cover 34 and the external, insulated protective barrier 36, which was the test subject in FIG. 60, is shown in FIGS. 61-63 . The test materials were returned via FedEx® shipping to the inventors and arrived 11 days after the cold test described in FIG. 60. FIG. 61 is a photograph of the space bag 1 inside of the foam cover 34 and the protective barrier 36 and closed by clasps 362 (zipping cable ties in this case). In FIG. 62, the space bag has been removed from the silicone protective barrier 36 and the foam cover 34 and the front of the branded space bag is visible. The rear view of the cold tested bag is shown in FIG. 63 and the mycelia are visible as white growth against the brown growth substrate background. The filter 11 is visible on the other side of the generator through the transparent membrane. Thereafter, viability analysis was commenced. In FIG. 63, the mycelial culture within the sample that was exposed to −80 C for 45 minutes appears dry and somewhat “freezer burned.” The apparatus appeared to withstand the cold test as it defrosted quickly, and the interior contents appeared to have resisted hard freezing. These visual observations would seem to imply that the mycelium most likely did feel some effects from the cold shock.

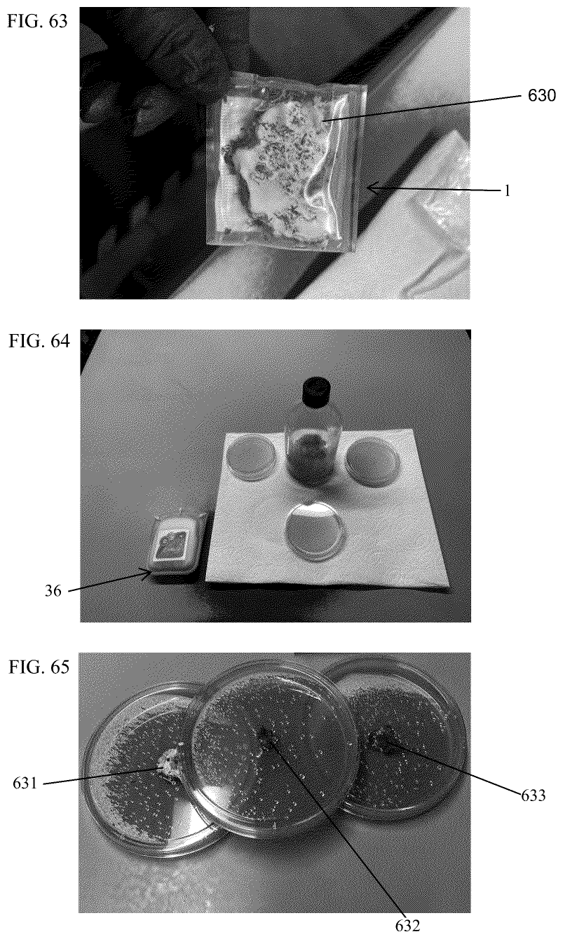

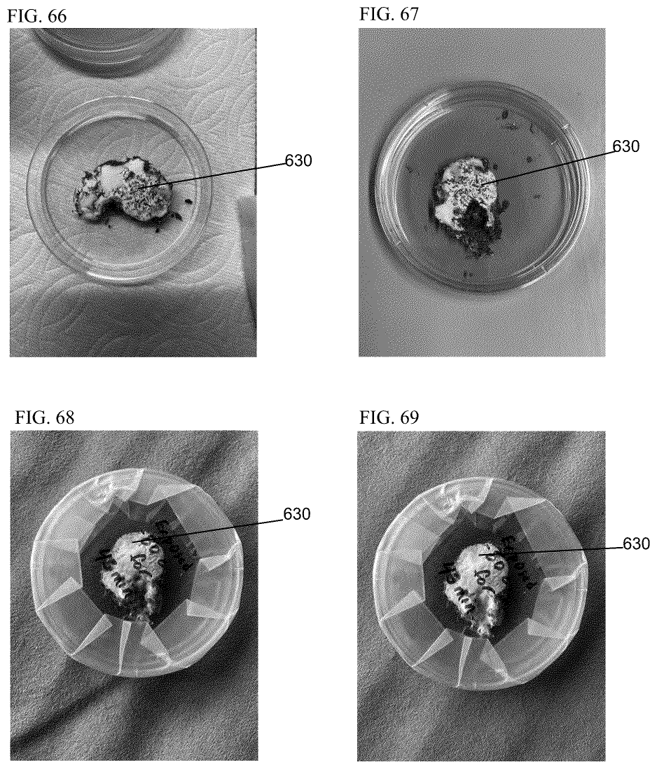

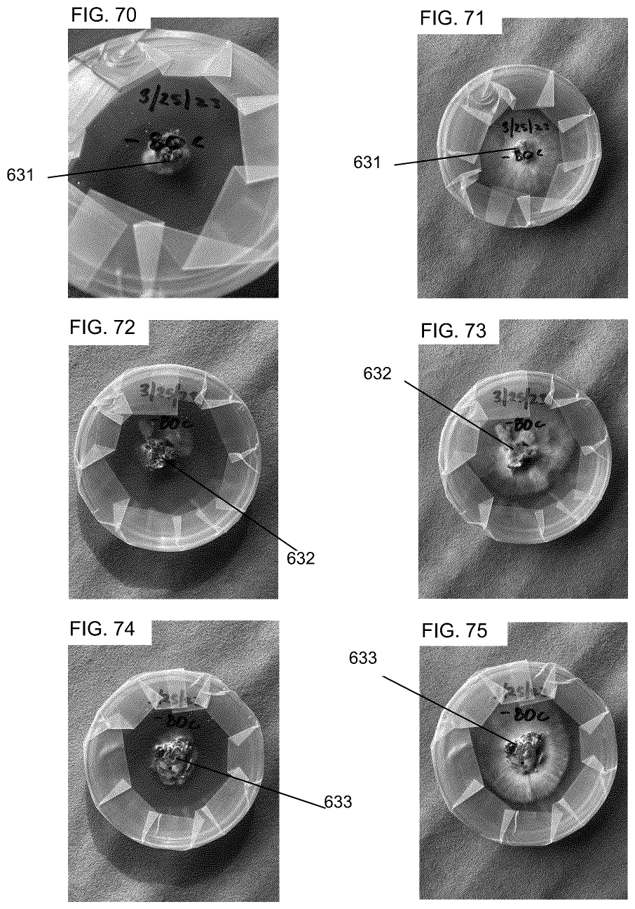

In FIGS. 64-75, the viability of the cold-tested mycelium exposed to cold within the 5-gram CO₂ generator was proven by the process of tissue culture testing. As shown in FIG. 64, three petri plates were prepared to receive tissue culture samples from the 5-gram cold-tested bag which was returned with the silicone jacket 36 still secured with the zip ties. These tissue culture testing procedures were commenced 1 day after the sample was returned, about 12 days after the organisms were exposed to cold. In FIG. 65, the three petri plates from FIG. 64 were photographed 48 hours after the tissue cultures 631, 632, 633 were transferred. The 5-gram test sample 630 is in the petri plate in FIG. 66. Then, FIG. 67 shows the sample 630 after the tissue cultures have been taken away. In FIG. 68, regrowth of the resected mycelia 630 is already visible after 48 hours in the form of whitish growth expansion as the mycelium progresses across the petri plates. In FIG. 69, increased regrowth of the 5-gram test sample 630 was observed after 6 days. In similar experiments, viability results will typically be observed within 72-96 hours, however, within 48 hours of commencing this tissue culture transfer test, visible signs of growth were evident in each of the 3 petri plates. As can be observed in FIGS. 70-75, petri plates 1 with sample 631 (FIGS. 70-71), 2 with sample 632 (FIGS. 72-73), and 3 with sample 633 (FIGS. 74-75) each demonstrate noticeable regrowth of the mycelia in the form of whitish expansion on the sections cut away from the 5-gram test sample 630 at time intervals of 48 hours (2 days) in FIGS. 70, 72, and 74 and 6 days in FIGS. 71, 73, and 75.

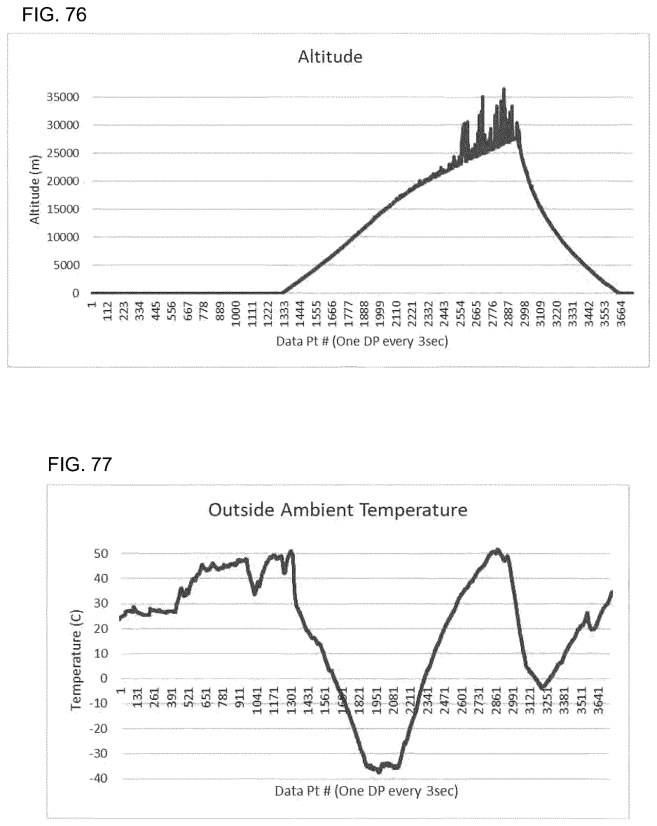

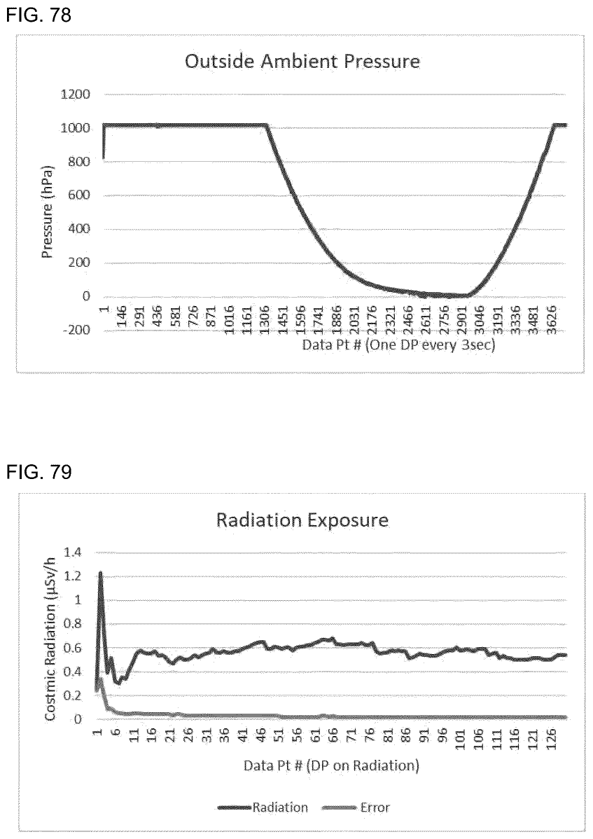

Once the launch occurred, real time survivability testing within extraterrestrial conditions was conducted to ensure viability of the mycelium for space based applications. FIG. 76 graphically demonstrates the altitude exposure experienced by the CO₂ generator during survivability testing. The x-axis indicates a series of numbered data points where the data points were collected every three seconds and number 1 through 3664. The y-axis shows altitude in meters (m), with lines demarcating 0 m, 5000 m, 10,000 m, 15,000 m, 20,000 m, 25,000 m, 30,000 m, and 35,000 m. The beginning and ending altitude measurements occurred at sea level (0 meters). The highest altitude of more than 35,000 meters was reached around data point 2887. FIGS. 77 and 78, respectively, graphically demonstrates the outside ambient temperature and outside ambient pressure present during the survivability testing. In FIG. 77, the graph shows temperature exposure in space. The x-axis sets out data points 1-3641 taken at three second intervals. The y-axis sets out temperatures in intervals of ten degrees beginning at −40 C and ending at 50 C. The temperatures experienced during the space flight ranged from about −38 C to over 50 C. The lowest temperature was reached around data point 1951 (which should amount to about one hour and thirty-seven minutes after launch). In FIG. 78, a graph shows the results of pressure and vacuum exposure experienced during the survivability test. The x-axis indicates data points between 1 and 3626, collected at intervals of three seconds each. The y-axis displays pressure indications in Hectopascals (hPa) with lines demarcating −200, 0, 200 400, 600, 800, 1,000, and 1,200 hPa. Near launch and upon return to earth, the pressure was 1,000 hPa, a typical reading for sea level. The generator subjected to survivability testing with just a silicone jacket 36 experienced vacuum conditions where the readings were effectively 0 hPa. FIG. 79 graphically demonstrates the radiation exposure experienced by the apparatus and mycelia during the survivability tests performed during the space flight. The upper line displays the results of the radiation testing while the bottom line demonstrates an error not relevant to the reported results here. The x-axis list data collection points taken at three second intervals and ranging from data point 1 to data point 126. The y-axis displays cosmic radiation in micro-Sieverts/hour (μSv/h) and demarcates the values of 0, 0.2, 0.4, 0.6, 0.8, 1.0, 1.2 and 1.4 μSv/h. This test shows that the test sample was exposed to a steady barrage of radiation throughout the test.

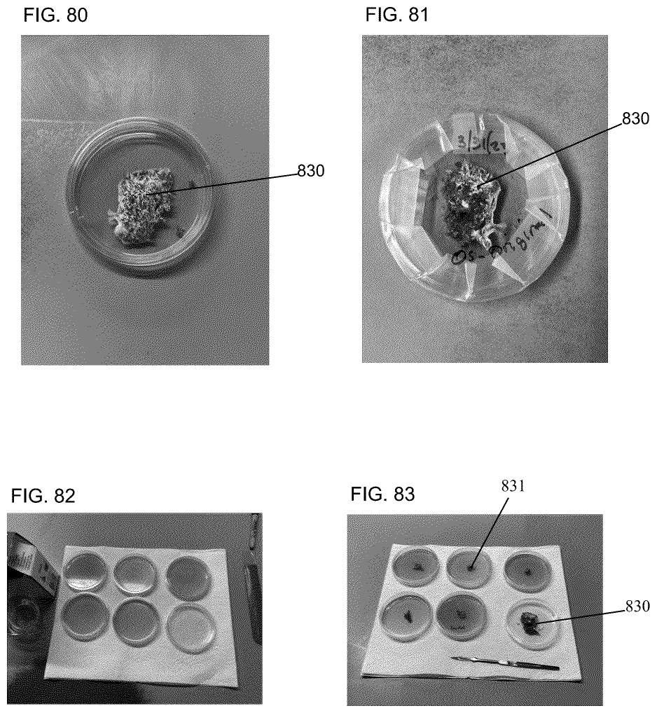

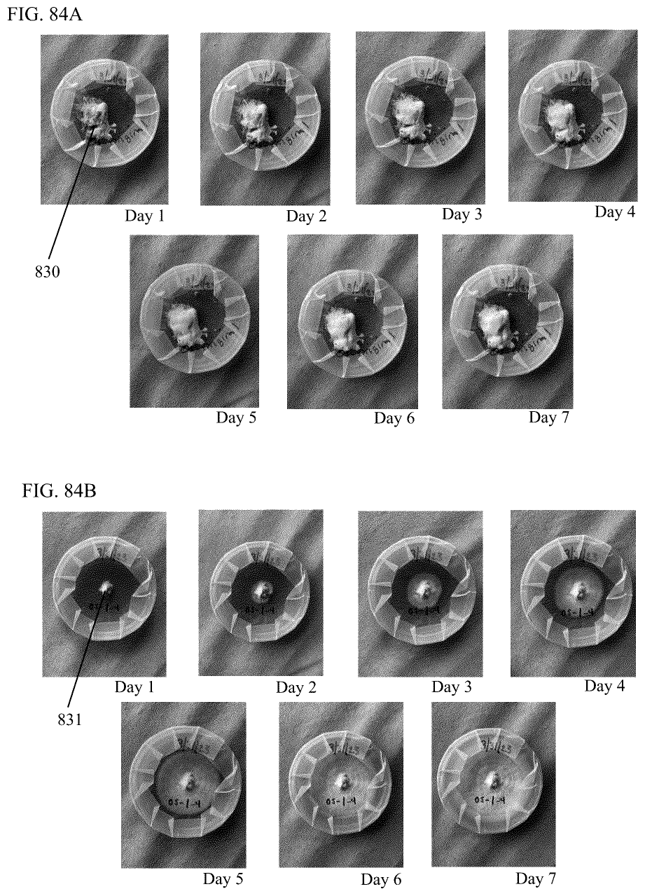

The test subject bags were returned after the space flight via FedEx to inventors for viability analysis. The external silicone pouch showed signs of having been exposed to the vacuum of space in that virtually all the air inside had been sucked out and the pouch was noticeably flat and compressed. Upon opening the silicone pouch the air pressure equalized immediately with an almost audible rush of air. The mycelial culture from the ECS externally-mounted 5-gram generator 830, after being removed from the silicone pouch 36 and thermal jacket 34, visually appeared to be alive and in good condition, see, e.g., FIG. 80. Viability of mycelium was again proven by the process of tissue culture testing beginning with the 5-gram sample 830 shown in FIG. 80 and removing sections of the sample as shown in FIG. 81. The tissue culture testing commenced with the materials shown in FIGS. 82-83 . In FIG. 82, five petri plates with growth medium were prepared to receive the tissue culture samples from the 5-gram CO₂ bag 830 (shown in the 6^{th} petri dish in FIG. 83). Tissue culture procedure was performed on the same day the sample was returned to the inventors. A selection of results are displayed in FIGS. 84A and 84B where cultures were visually monitored and photographically catalogued over seven days. FIG. 84A demonstrates the regrowth of the main sample 830 with the photos in sequential order showing day one in the top left and day seven in the bottom right. Then FIG. 84B demonstrates the regrowth of a cultured sample 831 from FIG. 83 again with the photos in sequential order from top left to bottom right showing seven days of regrowth. After only 48 hours, visible signs of growth in the form of whitish expansion were evident in all of the petri plates which received cultures from the 5-gram CO₂ bag which was externally mounted to the ECS and launched to more than 36,000 meters above sea level—see FIG. 76.

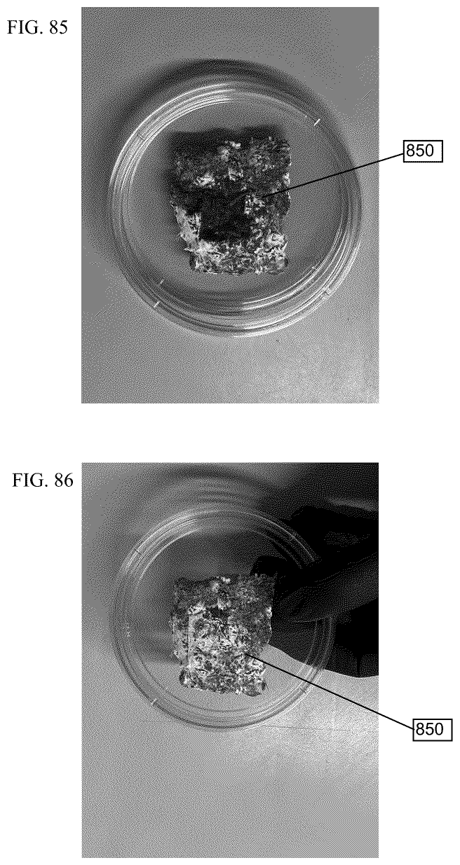

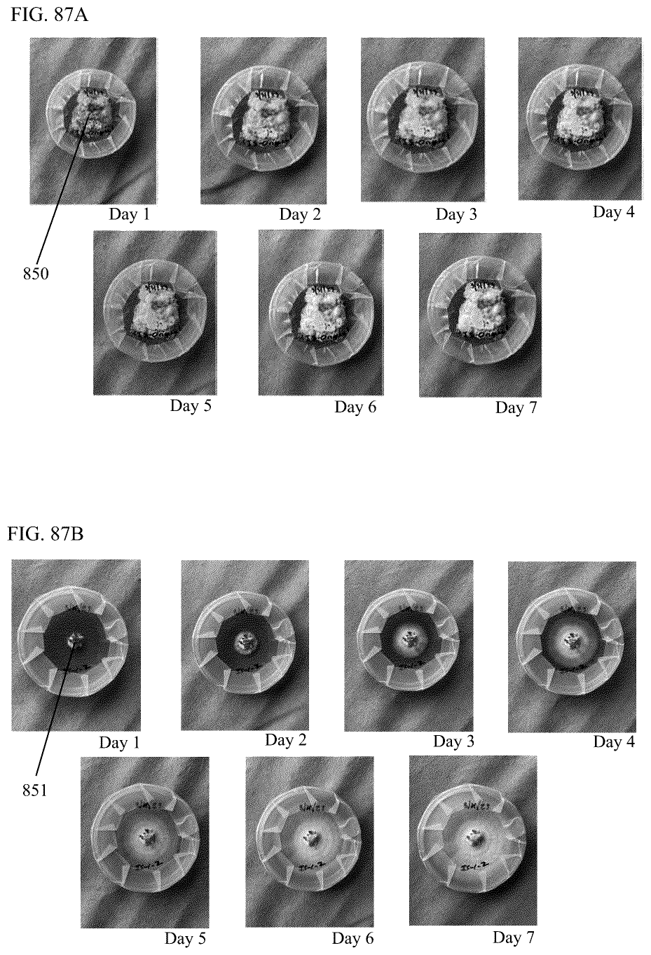

When the 10-gram generator which traveled within the ECS chamber (similar to the representation in FIG. 55) was returned from the space flight, it showed no signs of degradation. The mycelium visually showed signs of life activity with fresh hyphae being seen extending from the mycelial mass, see FIGS. 85-86 which show the 10-grams of mycelial mass 850 removed from the container. Further processing was performed with four petri plates receiving tissue culture samples from the internal 10-gram CO₂ bag. Tissue culture procedure was performed on the same day the sample was returned to the inventors. Cultures were monitored over seven days. Again, after only 48 hours, visible signs of growth in the form of whitish expansion were evident in all petri plates and a sampling of photographs of the results is shown in FIGS. 87A and 87B. FIG. 87A displays the photographs in sequential order from top left to bottom right to demonstrate regrowth of the mycelial mass 850. FIG. 87B displays a tissue culture 851 taken from the mycelial mass 850, and this series of photos taken on each of seven days is also in sequential order from top left to bottom right.

All petri plate cultures taken from the external 5-gram CO₂ bag and internal 10-gram CO2 bag have been sub-cultured and monitored for any noticeable changes that may have occurred from the direct exposure to altitude, temperature, pressure, and radiation elements of space and no ill effects have been observed. Further expansion of the mycelium testing may be conducted and CO2 output tests performed to ascertain the effectiveness of CO2 production compared to their earth bound counterparts.

This disclosure has described the organism-driven CO2 generating apparatus and methods for creating, implementing, and optimizing the apparatus. The example apparatus employs a floating, non-constrained interior filtered container placed in a secondary outside opposite opposed filtered container, both made to travel within terrestrial and extraterrestrial areas. The apparatus is intentionally designed to succeed in planetary environments as well as extraterrestrial environments whether they are inhabited areas or not. The disclosed apparatus can vary in size, or concentration, or proportion as the demands of the plants or environment may require. The present invention is particularly designed for highly controlled environmental systems (such as ECS) because it is a vapor equalization device which can collect condensation while it delivers gas across the filters. It requires no combustion or gas cartridges but is a self-contained, non-electrical, solid state emission system for gas supplementation. The size of the filter on the primary container will take up most of the surface area of at least one wall. The exterior container can be removed or vented for pressure differential without jeopardizing prepared growth media.

Reference throughout this specification to “one embodiment,” “an embodiment,” “one example,” or “an example,” “variation” or “implementation” means that a particular feature, structure, or characteristic described in connection with the embodiment or example is included in at least one embodiment of the present disclosure. Thus, appearances of the phrases “in one embodiment,” “in an embodiment,” “one example,” or “an example,” “variation” or “implementation” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

It is further intended that any other embodiments or implementations of the present invention which result from any changes in application or method of use or operation, method of manufacture, shape, size, or material which are not specified within the detailed written description or illustrations contained herein yet are considered apparent or obvious to one skilled in the art are within the scope of the present invention.

We claim:

1. An extraterrestrial CO₂ generator comprising:
a sealed container having an interior volume and configured to permit air exchange between the interior volume and an extraterrestrial environment,
a mycelium organism enclosed within the interior volume, and
a food source also enclosed within the interior volume.

2. The generator of claim 1, wherein an integral seal is disposed within the interior volume.

3. The generator of claim 2, wherein the integral seal separates the mycelium organism from the food source.

4. The generator of claim 3, wherein the integral seal is configured to be separated by external manipulation to allow combination of the mycelium organism and the food source without opening the sealed container.

5. The generator of claim 2, wherein the sealed container is constructed of a membranous material impervious to air transfer and a space below the integral seal is further comprised of an air-permeable filter.

6. The generator of claim 5, wherein the sealed container is placed within a separately sealed second container constructed of a membranous material impervious to air transfer and a second air-permeable filter.

7. The generator of claim 5, wherein the sealed container is placed within a separately sealed second container constructed of an air-permeable, membranous material.

8. The generator of claim 7, wherein the second container traps condensation away from the extraterrestrial environment.

9. The generator of claim 7, wherein the second container is fitted with a pressure relief valve.

10. The generator of claim 2, wherein the sealed container is constructed of an air-permeable, membranous material.

11. The generator of claim 10, wherein the sealed container is placed within a separately sealed second container entirely constructed of an air-permeable, membranous material.

12. The generator of claim 11, wherein the second container is fitted with a pressure relief valve.

13. The generator of claim 10, wherein the sealed container is placed within a separately sealed second container constructed of a membranous material impervious to air transfer having an air-permeable filter.

14. The generator of claim 1, wherein the container is entirely constructed of an air-permeable, membranous material.

15. The generator of claim 14, wherein the container is placed within a second container entirely constructed of an air-permeable membranous material.

16. The generator of claim 2, wherein the container is further configured for insertion into a sleeve.

17. The generator of claim 2, wherein the container is further configured for insertion into a protective barrier.

18. The generator of claim 17, wherein the container is first inserted into a sleeve.

19. The generator of claim 1, wherein a combined weight of the mycelium organism and food source is less than approximately 20 grams.

20. An extraterrestrial CO₂ generator comprising:
a respirating-organism growing environment having
a free-floating inner container sealed on all sides and having a first passive gas filter,
the free-floating inner container further comprising an internal press seal separating a respirating organism and a food substrate,
an external container separately sealed on all sides, having a second passive gas filter,
wherein, once the internal press seal is released, the respirating organism is joined with the food substrate within the free-floating inner container, carbon dioxide moves out of the first passive gas filter and out of the second passive gas filter and into the space environment while condensation is trapped within the external container.

21. The generator of claim 20, further comprising at least one receiver selected from the following group: a sealable case, a cover, and a protective jacket.

22. An extraterrestrial CO₂ generator comprising:
a respirating-organism growing environment having
a free-floating, air-permeable inner container sealed on all sides,
the free-floating inner container further comprising an internal press seal separating a respirating organism and a food substrate,
an external, air-permeable container separately sealed on all sides,
wherein, once the internal press seal is released, the respirating organism is joined with the food substrate within the free-floating inner container, carbon dioxide moves out of the inner container and out of the external container and into the space environment while condensation is trapped within the external container.

23. The generator of claim 22, further comprising at least one receiver selected from the following group: a sealable case, a cover, and a protective jacket.
